(19) United States
(12) Patent Application Publication
(10) Pub. No.: US 2009/0023801 A1
(43) Pub. Date: Jan. 22, 2009

(54) INHIBITORS OF BETA AMYLOID PRODUCTION

(75) Inventors: Thomas J. Caggiano, Morrisville, PA (US); Koi M. Morris, Plainsboro, NJ (US); Boyd L. Harrison, Princeton Junction, NJ (US); Anthony F. Kreft, III, Langhorne, PA (US); Dennis M. Kubrak, Philadelphia, PA (US); Dane M. Springer, Yardley, PA (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 12/172,505

(22) Filed: Jul. 14, 2008

Related U.S. Application Data
(60) Provisional application No. 60/959,675, filed on Jul. 16, 2007.

(51) Int. Cl. A61K 31/381; C07D 333/34; C07C 307/02; A61K 31/18

(52) U.S. Cl. 514/445; 549/65; 564/84; 514/604

(57) ABSTRACT

Novel sulfonamide compounds useful in the treatment of conditions related to the production of beta-amyloid are described, as are routes to their preparation. The sulfonamide compounds are of the following structure, wherein R₁-R₃ are defined herein. Also provided are pharmaceutical compositions containing these compounds and/or prodrugs of these compounds and a physiologically compatible carrier. These compounds are specifically useful for inhibiting beta amyloid production, and treating Alzheimer's Disease, amyloid angiopathy, cerebral amyloid angiopathy, systemic amyloidosis, hereditary cerebral hemorrhage with amyloidosis of the Dutch type, inclusion body myositis, mild cognitive impairment (MCI) and Down's syndrome.

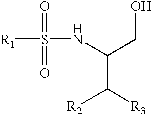

### CROSS-REFERENCE TO RELATED APPLICATIONS

This application claims the benefit of the priority of U.S. Provisional Patent Application No. 60/959,675, filed Jul. 16, 2007.

### BACKGROUND OF THE INVENTION

This invention relates to inhibitors of beta amyloid production, which have utility in the treatment of the effects of Alzheimer's disease.

Alzheimer's disease (AD) is the most common form of dementia (loss of memory) in the elderly. The main pathological lesions of AD found in the brain consist of extracellular deposits of beta amyloid protein in the form of plaques and angiopathy and intracellular neurofibrillary tangles of aggregated hyperphosphorylated tau protein. Evidence shows that elevated beta amyloid levels in the brain not only precede tau pathology but also correlate with cognitive decline. Further suggesting a causative role for beta amyloid in AD, studies have shown that aggregated beta amyloid is toxic to neurons in cell culture.

Beta amyloid protein is composed mainly of 39-42 amino acid peptides and is produced from a larger precursor protein called amyloid precursor protein (APP) by the sequential action of the proteases beta secretase and gamma secretase. Although rare, cases of early onset AD have been attributed to genetic mutations in APP that lead to an overproduction of either total beta amyloid protein or its more aggregation-prone 42 amino acid isoform. Furthermore, people with Down's syndrome possess an extra chromosome that contains the gene that encodes APP. These individuals have elevated beta amyloid levels and develop AD later in life.

Phenylsulfonamide and heterocyclic sulfonamide inhibitors of beta amyloid production have been described. See, U.S. Pat. Nos. 6,878,742; 6,610,734; and 7,166,622 and US Patent Application Publication Nos. US-2005/0196813 and US-2005/0171180. Fluoro-and trifluoroalkyl-containing heterocyclic and phenyl sulfonamide inhibitors of beta amyloid production have also been described. See, US Patent Application Publication Nos. US-2004/0198778 and US-2007/0249722.

There continues to be a need for compounds and compositions useful in inhibiting beta amyloid production and in the treatment of the symptoms of Alzheimer's disease.

### SUMMARY OF THE INVENTION

In one aspect, compounds of formula (I) are described, wherein R₁-R₃ are defined herein.

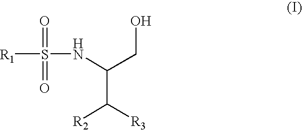

In another aspect, pharmaceutical compositions containing these compounds are described and contain a physiologically compatible carrier.

In a further aspect, pharmaceutical compositions containing prodrugs of the compounds described herein are described and contain a physiologically compatible carrier.

In yet a further aspect, methods of inhibiting beta amyloid production in a subject are described and include delivering a compound described herein.

In still another aspect, methods of treating Alzheimer's Disease, amyloid angiopathy, cerebral amyloid angiopathy, systemic amyloidosis, hereditary cerebral hemorrhage with amyloidosis of the Dutch type, inclusion body myositis, mild cognitive impairment (MCI) and Down's syndrome in a subject are described and include administering a compound described herein to the subject.

In a further aspect, pharmaceutical kits are described. The kits have a container which includes a pharmaceutical composition described herein.

In yet another aspect, methods are described for preparing a compound of formula (I).

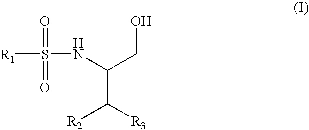

Other aspects and advantages of the invention will be readily apparent from the following detailed description of the invention.

### BRIEF DESCRIPTION OF THE DRAWINGS

FIG. 1. provides the powder X-ray diffraction pattern for a sample of 5-Chloro-N-[(1S *,2S *)-2-(3,5-difluorophenyl)-3,3,3-trifluoro-1-(hydroxymethyl)propyl]thiophene-2-sulfonamide prepared as described herein.

### DETAILED DESCRIPTION OF THE INVENTION

Haloalkyl-containing aryl or heteroarylsulfonamide derivatives of 1,2 amino alcohols of formula (I) are provided. These compounds are inhibitors of beta amyloid protein production from APP and are therefore useful for the treatment of physiological conditions associated with increased beta amyloid levels (e.g. AD, Down's syndrome). These compounds lower beta amyloid protein levels and are useful in patients susceptible to, or suffering from, diseases such as Alzheimer's disease, mild cognitive impairment and Down's syndrome. Lower beta amyloid protein levels resulting from administration of these compounds should reduce toxic beta amyloid aggregates in the brains of these patients.

Compounds of formula (I) are of the structure:

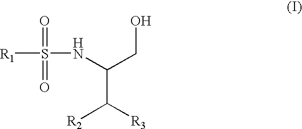

wherein, R₁ is aryl, substituted aryl, heteroaryl, or substituted heteroaryl; R₂ is haloalkyl or substituted haloalkyl; and R₃ is aryl, substituted aryl, heteroaryl, or substituted heteroaryl; or a pharmaceutically acceptable salt, prodrug, tautomer, or metabolite thereof.

In one embodiment, R₁ is an aryl or substituted aryl. In another embodiment, R₁ is a 6 to 14 membered unsaturated carbon-based ring or substituted 6 to 14 membered unsaturated carbon-based ring. In one example, R₁ is of the structure:

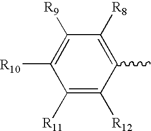

wherein, R⁸, R⁹, R¹⁰, R¹¹, and R¹² are independently selected from among H, halogen, C₁ to C₆ alkoxy, substituted C₁ to C₆ alkoxy, NO₂, C₁ to C₆ alkyl, substituted C₁ to C₆ alkyl, CN, C₁ to C₆ alkylcarbonyl, substituted C₁ to C₆ alkylcarbonyl, C₁ to C₆ alkylcarboxy, substituted C₁ to C₆ alkylcarboxy, CONH₂, CONH(C₁ to C₆ alkyl), CONH (substituted C₁ to C₆ alkyl), CON(C₁ to C₆ alkyl)₂, CON (substituted C₁ to C₆ alkyl)₂, S(C₁ to C₆ alkyl), S (substituted C₁ to C₆ alkyl), SO(C₁ to C₆ alkyl), SO (substituted C₁ to C₆ alkyl), SO₂(C₁ to C₆ alkyl), SO₂ (substituted C₁ to C₆ alkyl), NHSO₂(C₁ to C₆ alkyl), and NHSO₂ (substituted C₁ to C₆ alkyl); or R⁸ and R⁹; R⁹ and R¹⁰; R¹¹ and R¹²; or R¹⁰ and R¹¹ are fused to form (i) a saturated ring containing 3 to 8 carbon atoms; (ii) an unsaturated ring containing 5 to 8 carbon atoms; or (iii) a heterocyclic ring containing 1 to 3 heteroatoms selected from among O, N, and S in the backbone of the ring, wherein rings (i) to (iii) may be substituted by 1 to 3 substituents including C₁ to C₆ alkyl, substituted C₁ to C₆ alkyl, halogen, or CN. Desirably, R₁ is phenyl or substituted phenyl. More desirably, R₁ is a halogenated phenyl. Even more desirably, R₁ is 4-chlorophenyl.

In another embodiment, R₁ is a heteroaryl or substituted heteroaryl such as an unsaturated 5 or 6-membered ring having in its backbone 0 to 1 O or S-atoms and 0 to 4 N atoms, wherein the ring has at least one heteroatom in the backbone of the ring. In one example, R₁ is of the structure:

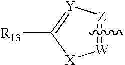

wherein, R₁₃ is selected from among H, halogen, and CF₃; W, Y and Z are independently selected from among C, CR₁₄ and N, wherein at least one of W, Y or Z is C; X is selected from among O, S, SO₂, and NR₁₅; R₁₄ is selected from among H, halogen, C₁ to C₆ alkyl, and substituted C₁ to C₆ alkyl; and R₁₅ is selected from among H, C₁ to C₆ alkyl, C₃ to C₈ cycloalkyl, SO₂(C₁ to C₆ alkyl), SO₂ (substituted C₁ to C₆ alkyl), SO₂aryl, SO₂substituted aryl, CO(C₁ to C₆ alkyl), CO (substituted C₁ to C₆ alkyl), CO aryl and CO substituted aryl. Desirably, R₁ is a thiophene or substituted thiophene. More desirably, R₁ is a halogenated thiophene. Even more desirably, R₁ is 2-chloro-thiophen-5-yl.

As defined above, the compounds herein require that R₂ include a haloalkyl or substituted haloalkyl. The term “haloalkyl” as used herein refers to an alkyl as defined below which contains at least one halogen bound to the alkyl group, i.e., the halogen may be bound to at least one carbon atom of the alkyl group. In one embodiment, a haloalkyl includes one, two, or three halogen atoms on at least one carbon atom of the alkyl chain, e.g., CH₂F, CF₂H, and CF₃. In another embodiment, one or more of the carbon atoms of the alkyl chain may be halogenated. In yet another embodiment, haloalkyl includes an alkyl that is substituted with one or more fluorine atoms. In a further embodiment, R₂ is —(CHₘX′ₙ)_{z}CHₚX′_{q}; m and n are, independently, 0 to 2, provided that m+n=2; p and q are, independently, 0 to 3, provided that p+q=3; z is 0 to 12; and X′ is halogen; provided that both n and q are not 0. Desirably, z is 0 to 5. More desirably, R₂ is CF₃.

In another embodiment, R₂ is —(CHₘ(R₅)_{y}X′ₙ)_{z}CHₚ(R₅)ₒX′_{q}; y, m, and n are, independently, 0 to 2, provided that y+m+n=2; o, p, and q are, independently, 0 to 3, provided that o+p+q=3; z is 0 to 12; X′ is halogen; provided that both n and q are not 0; and R₅ is halogen, CN, OH, NO₂, C₁ to C₆ alkyl, C₁ to C₆ substituted alkyl, C₂ to C₆ alkenyl, substituted C₂ to C₆ alkenyl, C₂ to C₆ alkynyl, C₂ to C₆ substituted alkynyl, amino, aryl, substituted aryl, heterocyclic, substituted heterocyclic, heteroaryl, substituted heteroaryl, C₁ to C₆ alkoxy, aryloxy, C₁ to C₆ alkylcarbonyl, C₁ to C₆ alkylcarboxy, or arylthio. Desirably, R₂ is (C₁ to C₅ alkyl)CF₃.

In a further embodiment, R₃ is an aryl or substituted aryl. Desirably, R₃ is phenyl or substituted phenyl. More desirably, R₃ is phenyl substituted with one or more halogen atoms. Even more desirably, R₃ is 3,5-difluoro-phenyl, 4-fluorophenyl, 3-fluorophenyl, or 4-chlorophenyl.

In one embodiment, a compound of formula (I) is provided, wherein R₁ is substituted phenyl or substituted thiophene; R₂ is CF₃; and R₃ is phenyl or phenyl substituted with one or more halogen atoms; provided that the carbon-atom attached to the sulfonamide nitrogen atom has S-stereochemistry and provided that the carbon atom attached to R₂ and R₃ has R-stereochemistry.

The compounds may contain one or more asymmetric carbon atoms and some of the compounds may contain one or more asymmetric (chiral) centers and may thus give rise to optical isomers and diastereomers. Thus, the compounds include such optical isomers and diastereomers; as well as racemic and resolved, enantiomerically pure stereoisomers; as well as other mixtures of the R and S stereoisomers, and pharmaceutically acceptable salts, hydrates, and prodrugs thereof. These diastereomers may be separated using techniques known to those skilled in the art. Most conveniently, the diastereomers are separated using chiral, preparatory liquid chromatography. In one embodiment, the compounds described herein have R-stereochemistry at the carbon atom attached to R₂ and R₃. In another embodiment, the compounds have S-stereochemistry at the carbon bearing the sulfonamide nitrogen atom. In a further embodiment, the compounds described herein have R-stereochemistry at the carbon atom attached to R₂ and R₃ and S-stereochemistry at the carbon bearing the sulfonamide nitrogen atom.

The compounds may encompass tautomeric forms of the structures provided herein characterized by the bioactivity of the drawn structures. Further, the compounds may also be used in the form of salts derived from pharmaceutically or physiologically acceptable acids, bases, alkali metals and alkaline earth metals.

Pharmaceutically acceptable salts can be formed from organic and inorganic acids including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids.

Pharmaceutically acceptable salts may also be formed from inorganic bases, desirably alkali metal salts including, e.g., sodium, lithium, or potassium, such as alkali metal hydroxides. Examples of inorganic bases include, without limitation, sodium hydroxide, potassium hydroxide, calcium hydroxide, and magnesium hydroxide. Pharmaceutically acceptable salts may also be formed from organic bases, such as ammonium salts, mono-, di-, and trimethylammonium, mono-, di- and triethylammonium, mono-, di- and tripropylammonium (iso and normal), ethyldimethylammonium, benzyldimethylammonium, cyclohexylammonium, benzylammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1-n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmonoethanolammonium, diethanolamine, ethylenediamine, and the like. In one embodiment, the base is selected from among sodium hydroxide, lithium hydroxide, potassium hydroxide, and mixtures thereof.

These salts, as well as other compounds, can be in the form of esters, carbamates and other conventional “pro-drug” forms, which, when administered in such form, convert to the active moiety in vivo. In one embodiment, the prodrugs are esters. In another embodiment, the prodrugs are carbamates. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996), which is incorporated by reference.

The compounds discussed herein also encompass “metabolites” which are unique products formed by processing the compounds by the cell or subject. Desirably, metabolites are formed in vivo.

The term “alkyl” is used herein to refer to both straight- and branched-chain saturated aliphatic hydrocarbon groups. In one embodiment, an alkyl group has 1 to about 10 carbon atoms (i.e., C₁, C₂, C₃, C₄, C₅ C₆, C₇, C₈, C₉, or C₁₀ ). In another embodiment, an alkyl group has 1 to about 6 carbon atoms (i.e., C₁, C₂, C₃, C₄, C₅ or C₆). In a further embodiment, an alkyl group has 1 to about 4 carbon atoms (i.e., C₁, C₂, C₃, or C₄).

The term “alkenyl” is used herein to refer to both straight- and branched-chain alkyl groups having one or more carbon-carbon double bonds. In one embodiment, an alkenyl group contains 2 to about 10 carbon atoms (i.e., C₂, C₃, C₄, C₅, C₆, C₇, C₈, C₉, or C₁₀). In another embodiment, an alkenyl group has 1 or 2 carbon-carbon double bonds and 2 to about 6 carbon atoms (i.e., C₂, C₃, C₄, C₅ or C₆).

The term “alkynyl” is used herein to refer to both straight- and branched-chain alkyl groups having one or more carbon-carbon triple bonds. In one embodiment, an alkynyl group has 2 to about 10 carbon atoms (i.e., C₂, C₃, C₄, C₅, C₆, C₇, C₈, C₉, or C₁₀). In another embodiment, an alkynyl group contains 1 or 2 carbon-carbon triple bonds and 2 to about 6 carbon atoms (i.e., C₂, C₃, C₄, C₅, or C₆).

The term “cycloalkyl” is used herein to refer to cyclic, saturated aliphatic hydrocarbon groups. The term cycloalkyl may include a single ring or two or more rings fused together to form a multicyclic ring structure. A cycloalkyl group may thereby include a ring system having 1 to about 5 rings. In one embodiment, a cycloalkyl group has 3 to about 14 carbon atoms (i.e., C₃, C₄, C₅, C₆, C₇, C₈, C₉, C₁₀, C₁₁, C₁₂, C₁₃, or C₁₄). In another embodiment, a cycloalkyl group has 3 to about 6 carbon atoms (i.e., C₃, C₄, C₅ or C₆).

The terms “substituted alkyl”, “substituted alkenyl”, “substituted alkynyl”, and “substituted cycloalkyl” refer to alkyl, alkenyl, alkynyl, and cycloalkyl groups, respectively, having one or more substituents including, without limitation, hydrogen, halogen, CN, OH, NO₂, amino, aryl, heterocyclic, heteroaryl, alkoxy, aryloxy, alkylcarbonyl, alkylcarboxy, alkylamino, and arylthio.

The term “substituted haloalkyl” refers to a haloalkyl having one or more substituents on the alkyl moiety including, without limitation, hydrogen, halogen, CN, OH, NO₂, amino, aryl, heterocyclic, heteroaryl, alkoxy, aryloxy, alkylcarbonyl, alkylcarboxy, alkylamino, and arylthio.

The term “arylthio” as used herein refers to the S(aryl) group, where the point of attachment is through the sulfur-atom and the aryl group can be substituted as noted above.

The term “alkoxy” as used herein refers to the O(alkyl) group, where the point of attachment is through the oxygen-atom and the alkyl group can be substituted as noted above.

The term “aryloxy” as used herein refers to the O(aryl) group, where the point of attachment is through the oxygen-atom and the aryl group can be substituted as noted above.

The term “alkylcarbonyl” as used herein refers to the C(O)(alkyl) group, where the point of attachment is through the carbon-atom of the carbonyl moiety and the alkyl group can be substituted as noted above.

The term “alkylcarboxy” as used herein refers to the C(O)O(alkyl) group, where the point of attachment is through the carbon-atom of the carboxy moiety and the alkyl group can be substituted as noted above.

The term “alkylamino” as used herein refers to both secondary and tertiary amines where the point of attachment is through the nitrogen-atom and the alkyl groups can be substituted as noted above. The alkyl groups can be the same or different.

The term “halogen” as used herein refers to Cl, Br, F, or I groups.

The term “aryl” as used herein refers to an aromatic, carbocyclic system, e.g. of about 5 to 20 carbon atoms, which can include a single ring or multiple unsaturated rings fused or linked together where at least one part of the fused or linked rings forms the conjugated aromatic system. An aryl group may thereby include a ring system having 1 to about 5 rings. The aryl groups include, but are not limited to, phenyl, naphthyl, biphenyl, anthryl, tetrahydronaphthyl, phenanthryl, indene, benzonaphthyl, and fluorenyl.

The term “substituted aryl” refers to an aryl group which is substituted with one or more substituents including halogen, CN, OH, NO₂, amino, alkyl, cycloalkyl, alkenyl, alkynyl, alkoxy, C₁ to C₃ perfluoroalkyl, C₁ to C₃ perfluoroalkoxy, aryloxy, alkylcarbonyl, alkylcarboxy, —C(NH₂)═N—OH, —SO₂—(C₁to C₁₀ alkyl), —SO₂—(C₁ to C₁₀ substituted alkyl), —O—CH₂-aryl, alkylamino, arylthio, aryl, or heteroaryl, which groups can be substituted. Desirably, a substituted aryl group is substituted with 1 to about 4 substituents.

The term “heterocycle” or “heterocyclic” as used herein can be used interchangeably to refer to a stable, saturated or partially unsaturated 3- to 20-membered monocyclic or multicyclic heterocyclic ring. The heterocyclic ring has carbon atoms and one or more heteroatoms including nitrogen, oxygen, and sulfur atoms in its backbone. In one embodiment, the heterocyclic ring has 1 to about 4 heteroatoms in the backbone of the ring. When the heterocyclic ring contains nitrogen or sulfur atoms in the backbone of the ring, the nitrogen or sulfur atoms can be oxidized. Further, when the heterocyclic ring contains nitrogen atoms, the nitrogen atoms may optionally be substituted with H, C₁ to C₆ alkyl, substituted C₁ to C₆ alkyl, CO₂ (C₁ to C₆ alkyl), SO₂(C₁ to C₆ alkyl), SO₂ (substituted C₁ to C₆ alkyl), SO₂aryl, SO₂ substituted aryl, CO(C₁ to C₆ alkyl), CO (substituted C₁ to C₆ alkyl), CO aryl or CO substituted aryl. The heterocyclic ring can be attached through a heteroatom or carbon atom provided the resultant heterocyclic ring structure is chemically stable. When the heterocyclic ring is a multicyclic ring, it may contain 2, 3, 4, or 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 20-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. Further, when the heteroaryl ring contains nitrogen atoms, the nitrogen atoms may optionally be substituted with H, C₁ to C₆ alkyl, substituted C₁ to C₆ alkyl, CO₂ (C₁ to C₆ alkyl), SO₂ (C₁ to C₆ alkyl), SO₂ (substituted C₁ to C₆ alkyl), SO₂aryl, SO₂substituted aryl, CO(C₁ to C₆ alkyl), CO (substituted C₁ to C₆ alkyl), CO aryl, or CO substituted aryl. The heteroaryl ring can be attached through a heteroatom or carbon atom provided the resultant heterocyclic ring structure is chemically stable. When the heteroaryl ring is a multicyclic heteroatom-containing ring, it may contain 2, 3, 4, or 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents including halogen, CN, OH, NO₂, amino, alkyl, cycloalkyl, alkenyl, alkynyl, C₁ to C₃ perfluoroalkyl, C₁ to C₃ perfluoroalkoxy, alkoxy, aryloxy, alkylcarbonyl, alkylcarboxy, —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl, may be optionally substituted. A substituted heterocycle or heteroaryl group may have 1, 2, 3, or 4 substituents.

The compounds of formula (I) may be prepared following the descriptions and Schemes illustrated below.

### Synthesis of the Compounds

The compounds of formula (I) may be prepared via several routes using techniques and reagents that are well known to one skilled in the art of organic synthesis. The compounds may therefore be prepared using the methods described below, together with synthetic methods known in the synthetic organic arts or variations of these methods by one skilled in the art.

In the method summarized in Scheme 1, the compounds of formula (I) may be prepared by first reacting a halogenated acetophenone, a first base, and a tri-alkyl phosphonoacetate to form an α,β-unsaturated ester. In one example, the halogenated acetophenone is R₂C(O)R₃ (compound A), wherein R₂ and R₃ are defined above. In another example, the halogenated acetophenone is compound A1, wherein R₃ is defined above.

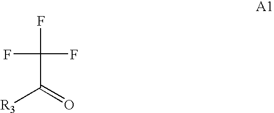

The first base utilized to prepare the α,β-unsaturated ester may be selected by one of skill in the art. Examples of bases that may be utilized include the bases described in W. S. Wadsworth in Organic Reactions 25: 73-253 (1977), which is hereby incorporated by reference. Typically, the first base is sodium hydride or tetramethylguanidine. Desirably, the tri-alkyl phosphonoacetate is (R₆O)₂P(O)CH₂CO₂R₇, wherein R₆ is C₁ to C₆ alkyl or substituted alkyl and R₇ is C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, phenyl, or substituted phenyl. In one embodiment, R₇ is benzyl or substituted benzyl. Typically, the reaction is performed in a solvent including, without limitation, tetrahydrofuran. However, other solvents may be utilized and include those described in Wadsworth cited above and hereby incorporated by reference. By doing so, α,β-unsaturated ester B is prepared, wherein, R₂, R₃ and R₇ are defined above.

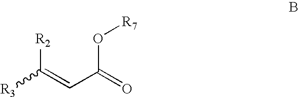

In another example, α,β-unsaturated ester B1 is prepared, wherein R₃ and R₇ are defined above.

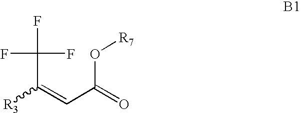

The α,β-unsaturated ester is then reduced to a saturated ester using techniques known to those of skill in the art including, without limitation, catalytic hydrogenation. In one embodiment, the hydrogenation is performed using hydrogen gas in the presence of a metal catalyst. A variety of metal catalysts may be utilized and include those described in S. Nishimura in Handbook of Heterogeneous Hydrogenation for Organic Synthesis; Wiley-Interscience: New York, 2001, pages 93-94, which is hereby incorporated by reference. In one example, saturated ester C is prepared via the reduction, wherein R₂, R₃ and R₇ are defined above.

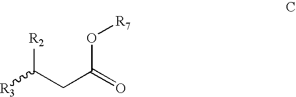

In another example, saturated ester C1 is prepared via the reduction, wherein R₃ and R₇ are defined above.

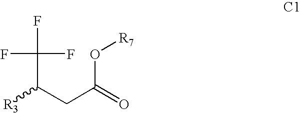

The saturated ester is then converted to an enolate. Typically, conversion to the enolate is performed using an alkali metal (M) amide base, alkali metal hydride, or alkali metal alkoxide. Desirably, the alkali metal amide base is a lithium amide base. In one embodiment, the lithium amide base is lithium diisopropylamide (LDA), lithium dicyclohexyl amide, lithium diethylamide, lithium dimethylamide, or lithium bis(trimethylsilyl)amide. In another embodiment, the alkali metal hydride is sodium hydride or potassium hydride, among others. In a further embodiment, the alkali metal alkoxide is potassium t-butoxide, among others. Conversion to the enolate is performed in the presence of an inert solvent. The term “inert” solvent as used herein refers to any organic solvent that does not react or interfere with any chemical reagents in the reaction mixture. Such an inert solvent for use in the preparation of the enolate may be selected by one of skill in the art and includes, without limitation, tetrahydrofuran (THF), diethylether, glyme, methyl t-butylether, (MTBE), or dioxane, among others. In one example, the enolate is compound D, wherein R₂, R₃, and R₇ are defined above and M is an alkali metal ion transferred from the lithium amide base.

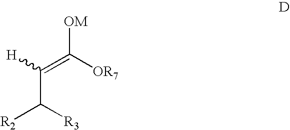

In another example, the enolate is compound D1, wherein R₃ and R₇ are defined above and M is an alkali metal ion transferred from the lithium amide base.

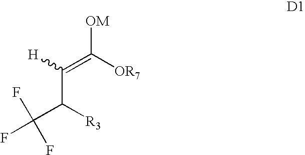

The enolate is then converted to azido-ester E, wherein R₂, R₃ and R₇ are defined above. The azido-ester is typically prepared using an azide transfer agent, which may readily be selected by one of skill in the art. A variety of azide transfer agents may be selected by one of skill in the art and include those described in D. A. Evans and T. C. Britton, J. Am. Chem. Soc. 109: 6881-6883, 1987, which is hereby incorporated by reference. In one embodiment, the azide transfer agent is triisopropylbenzenesulfonyl azide.

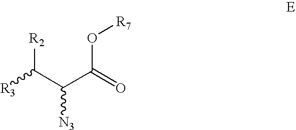

In another example, azido-ester E1 is prepared, wherein R₃ and R₇ are defined above.

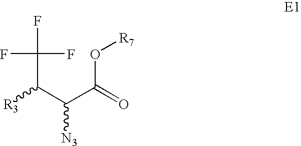

The azido-ester is then reduced to amino-ester F, wherein R₂, R₃ and R₇ are defined above. The reduction may be performed using techniques and reagents known to those of skill in the art. In one embodiment, the reduction is performed using catalytic hydrogenation. Suitable reagents for use in the reduction may be selected by one of skill in the art and include those described in R. Larock in Comprehensive Organic Transformations; Wiley-VCH: New York, 1999, pages 815-820, which is hereby incorporated by reference. Typically, the catalytic hydrogenation is performed using hydrogen gas and a metal catalyst as described above.

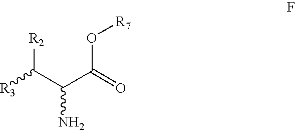

In another example, amino-ester F1 is formed from the reduction, wherein R₃ and R₇ are defined above.

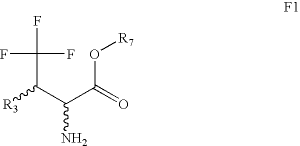

The amino-ester is then sulfonylated to a sulfonamido-ester. In one embodiment, the sulfonylation is performed using a sulfonyl chloride or sulfonic anhydride, among others. In another embodiment, the sulfonylation is performed as described in U.S. Pat. Nos. 6,610,734; 6,878,742; and 7,166,622 and US Patent Application Publication No. US-2004/0198778, which are incorporated by reference in their entirety. In one embodiment, the sulfonylation is performed using a sulfonylating agent such as the following, wherein R₈-R₁₃, W, X, Y, and Z are defined above and LG is a leaving group. The term “leaving group” as used herein refers to a chemical moiety that is displaced from a first chemical upon reaction of the first chemical with a second chemical. Examples of leaving groups that may be displaced from the sulfonylating agent include halogen atoms, such as chlorine or fluorine or sulfonates (e.g., mesylates, tosylates, triflates), among others.

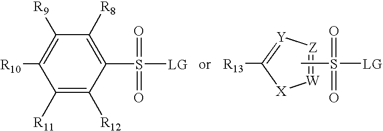

In one example, the sulfonamide-ester is compound G, where R₁-R₃, and R₇ are defined above

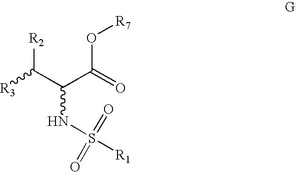

In another example, sulfonamide-ester compound G1 is prepared, wherein R₁, R₃ and R₇ are defined above.

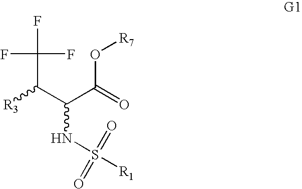

The sulfonamide-ester is then reduced to the compound of formula (I) using techniques known to those of skill in the art. The reduction may be performed using the transformations and reagents described in R. Larock in Comprehensive Organic Transformations; Wiley-VCH: New York, 1999, pages 1117-1120, which is hereby incorporated by reference. Desirably, the sulfonamido-ester is reduced using lithium borohydride.

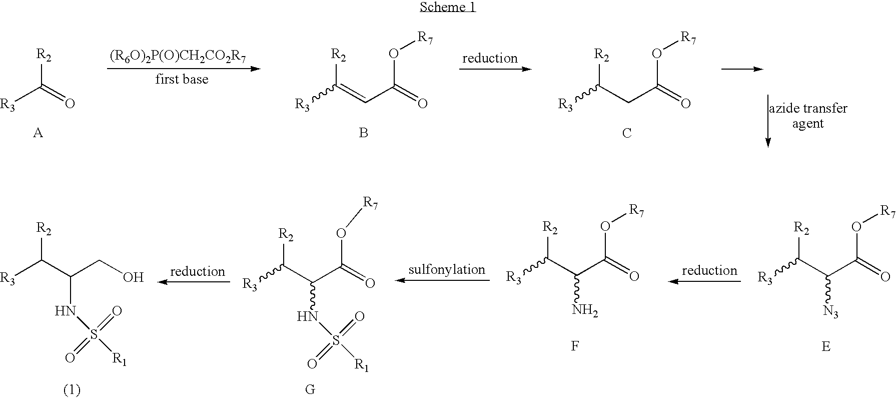

An example of a method for preparing the compounds described herein is described in Scheme 2. In this example, trifluoromethyl acetophenone A1 is reacted with a tri-alkyl phosphonoacetate in the presence of sodium hydride in THF to afford compound B1. Catalytic hydrogenation affords ester C1. The ester is converted to an enolate using lithium diisopropylamide in THF. The enolate is reacted with triisopropylbenzenesulfonyl azide to yield azide E1. The azide group of azide E1 is reduced by catalytic hydrogenation to produce amine F1. Sulfonylation of amine F1 using a sulfonyl chloride in the presence of a base and solvent provides sulfonamide GI. The ester group of sulfonamide G1 is then reduced to the compound (I) using lithium borohydride in THF. The mixture of alcohols obtained is most conveniently separated by chiral, preparative, liquid chromatography.

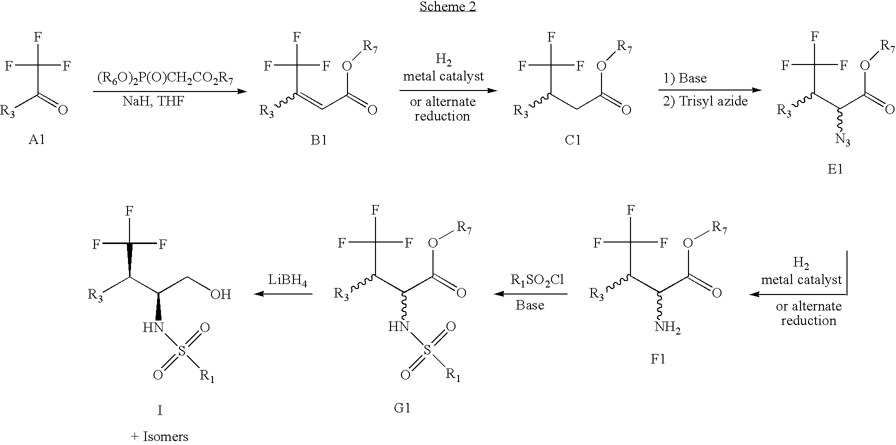

Another method for preparing the compounds of formula (I) is provided in Scheme 3 and first includes hydrolyzing an α,β-unsaturated ester to an α,β-unsaturated carboxylic acid. In one example, the α,β-unsaturated ester is compound B. In another example, α,β-unsaturated ester B1 is hydrolyzed. The hydrolysis may be performed using the reagents and conditions described in R. Larock in Comprehensive Organic Transformations; Wiley-VCH: New York, 1999, pages 1959-1968, which is hereby incorporated by reference. In one embodiment, the hydrolysis is performed in the presence of a base, which may readily be selected by one of skill in the art. Typically, the hydrolysis is performed in the presence of water. In one example, the α,β-unsaturated carboxylic acid prepared from the hydrolysis is compound H, wherein R₂ and R₃ are defined above.

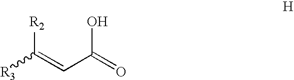

In a further example, α,β-unsaturated carboxylic acid H1 is prepared, wherein R₃ is defined above.

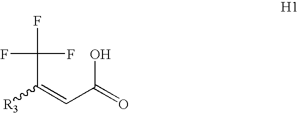

The α,β-unsaturated carboxylic acid is then converted to a mixed anhydride using techniques known to those of skill in the art including the reagents and conditions described in A. Beckwith in The Chemistry of Amides, J. Zabicky, Ed., Interscience Publishers: New York, 1970, page 91, which is hereby incorporated by reference. Typically, the conversion is performed using R₁₆COX″, wherein R₁₆ is C₁ to C₆ alkyl or substituted C₁ to C₆ alkyl and X″ is F, Cl, Br, I, or a carboxylate. In one embodiment, the carboxylate is, without limitation, a trimethylacetate, isovalerate, or diphenylacetate. In one example, R₁₆COX″ is an acyl chloride such as trimethylacetyl chloride, isovaleroyl chloride or diphenylacetyl chloride. The conversion is desirably performed in the presence of a second base. A variety of second bases may be selected by one of skill in the art and may include, without limitation, a tertiary amine such as triethylamine, among others. In one example, the mixed anhydride is compound J, wherein R₂, R₃, and R₁₆ are defined above.

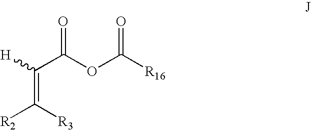

In another example, mixed anhydride J1 is prepared, wherein R₃ and R₁₆ are defined above.

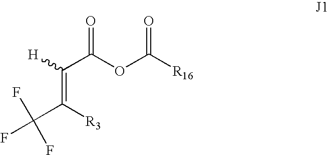

The mixed anhydride is then reacted with a nucleophile containing a chiral auxiliary. The phrase “nucleophile containing a chiral auxiliary” as used herein refers to a chemical compound that contains a chiral auxiliary. The nucleophile containing a chiral auxiliary desirably reacts with a second chemical compound and directs formation of chirality at one or more substituents in the second chemical compound. The nucleophile containing a chiral auxiliary may be added as a separate reagent or may be generated in situ immediately prior to reaction with the mixed anhydride. The mixed anhydride can be treated with various nucleophiles containing chiral auxiliaries. In one embodiment, the nucleophile containing a chiral auxiliary is an oxazolidinone containing chemical compound or imidazolidinone containing compound. In a further embodiment, the nucleophile containing a chiral auxiliary is selected from among the following:

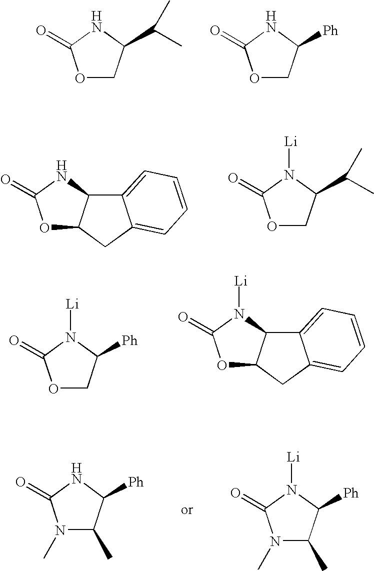

In another embodiment, the nucleophile containing a chiral auxiliary is lithium (S)-4-benzyloxazolidin-2-one.

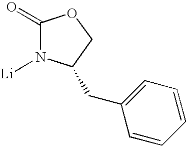

In one example, the reaction between the nucleophile containing a chiral auxiliary and the mixed anhydride provides compound K, wherein R₂ and R₃ are defined above.

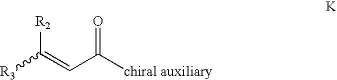

In a further example, the reaction between the nucleophile containing a chiral auxiliary and the mixed anhydride provides compound K1, wherein R₂ and R₃ are defined above.

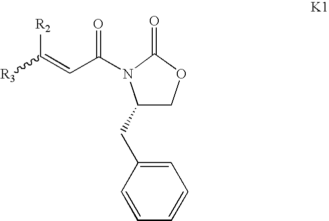

In another example, compound K2 is prepared via the reaction of the mixed anhydride with the nucleophile containing a chiral auxiliary, wherein R₃ is defined above.

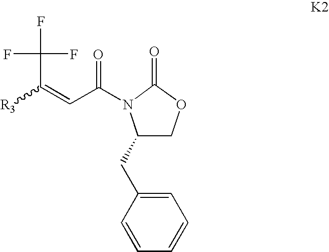

The compound containing the chiral auxiliary is then reduced using techniques known to those of skill in the art including, without limitation, catalytic hydrogenation. The reduction may be performed using reagents and conditions known to those skill in the art including those in S. Nishimura cited above and incorporated by reference herein. Desirably, the reduction is performed using hydrogen gas and a metal catalyst. In one example, compound L, wherein R₂ and R₃ are defined above, is formed,

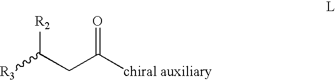

In another example, compound L1, wherein R₂ and R₃ are defined above, is formed.

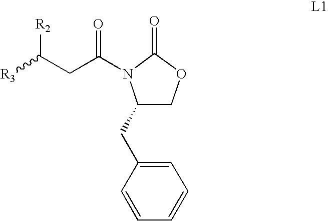

In a further example, compound L2 is prepared following the reduction, wherein R₃ is defined above.

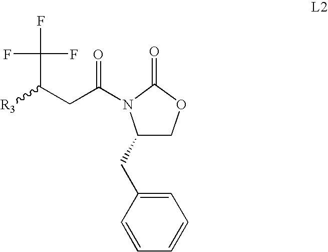

The reduced compound is then reacted with a base, which may readily be selected by one of skill in the art, including potassium hexamethyldisilazide and lithium amide bases such as LDA, among others. Other suitable bases may be selected from those described in D. A. Evans cited above and incorporated by reference. The reaction is desirably performed in an inert solvent such as THF, diethylether, glyme, methyl t-butylether or dioxane, among others. Additional solvents useful in this step may be selected by those skill in the art as provided in D. A. Evans cited above and incorporated by reference. The compound produced therefrom is then converted to an azido-imide, typically using an azide transfer agent. One of skill in the art would readily be able to select a suitable azide transfer agent including, without limitation, triisopropylbenzenesulfonyl azide. Other suitable azide transfer agents may be utilized including those recited in D. A. Evans et al. cited above and incorporated by reference. The reaction to form the azido-imide is desirably terminated using an acidic quench. One of skill in the art would readily be able to select suitable reagents to quench the reaction. Suitable reagents may include, without limitation, aqueous acids such as acetic acid or those described in D. A. Evans cited above and incorporated by reference.

The azido-imide is typically prepared as a mixture of predominately two diastereomers. In fact, the stereocenter of the carbon bearing the azide group is largely controlled by the choice of chiral auxiliary in the azidation substrate. Specifically, the use of the (S)-benzyl oxazolidinone generated predominately the (S) configuration of the carbon bearing the azide. In one example, azido-imide M, wherein R₂ and R₃ are defined above is prepared.

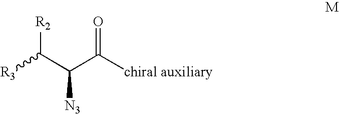

In a further example, azido-imide M1, wherein R₂ and R₃ are defined above is prepared.

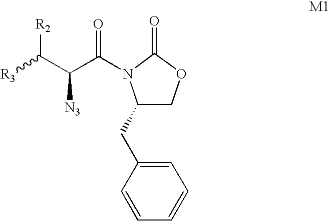

In another example, azido-imide compound M2, wherein R₃ is defined above is prepared.

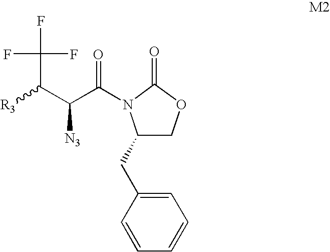

The azido-imide is then reduced to an amino-imide salt using reduction techniques known in the art including the transformation and reagents described in S. Nishimura in Handbook of Heterogeneous Hydrogenation for Organic Synthesis; Wiley-Interscience: New York, 2001, pages 377-379. Typically, the reduction is performed by catalytic hydrogenation, among others. In one embodiment, the hydrogenation is performed using hydrogen gas and a metal catalyst. The reduction is also performed in the presence of a proton source. The term “proton source” as used herein refers to a chemical compound that is either capable in itself of protonating the amine group or, by reaction with the solvent, generating an agent capable of protonating the amine group, thereby preventing unwanted side reactions. In one embodiment, the proton source is an inorganic acid, such as hydrochloric acid, or an acyl halide, such as propionyl chloride, among others. Desirably, the reduction is performed in the presence of hydrochloric acid or propionyl chloride. In one example, the amino-imide prepared from the reduction is compound N, wherein R₂ and R₃ are defined above and X₁ is a counterion derived from an organic carboxylic acid or inorganic acid. In one embodiment, X₁ is halogen, such as Cl, Br, I, or F, sulfonate, such as mesylate, tosylate, or triflate.

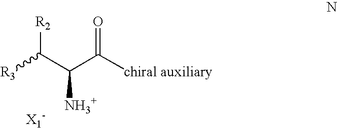

In another example, the amino-imide is compound N1, wherein R₂, R₃, and X₁ are defined above.

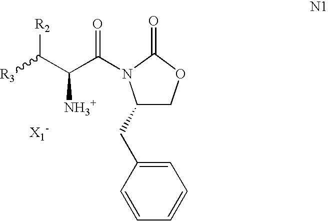

In another example, the amino-imide is compound N2, wherein R₃ and X₁ are defined above.

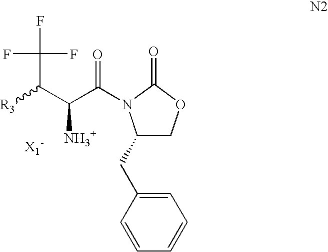

In a further example, the amino-imide is compound N3, wherein R₃ is defined above.

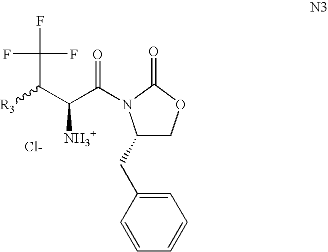

The amino-imide is then sulfonylated to form a sulfonamido-imide. The sulfonylation is typically performed using techniques known in the art as discussed above. In one embodiment, the sulfonylation is performed using a sulfonyl chloride or sulfonic anhydride. In one example, sulfonamido-imide P is prepared, wherein R₁-R₃ are defined above.

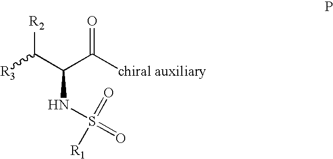

In another example, sulfonamide-imide P1 is prepared, wherein R₁-R₃ are defined above.

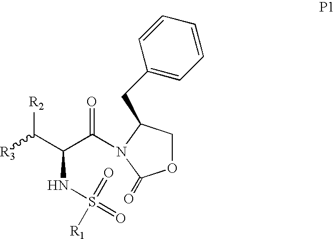

In another example, sulfonamide-imide P2 is prepared, wherein R₁ and R₃ are defined above.

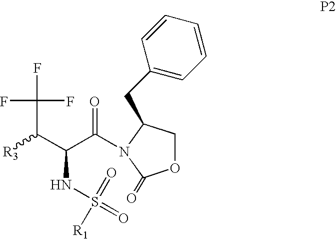

The sulfonamido-imide is then reduced to the compound of formula (I) using techniques and reagents known to those of skill in the art including, without limitation, lithium borohydride, lithium aluminum hydride or diisobutylaluminum hydride in a suitable solvent such as THF. However, selection of the reducing agent and solvent is within the capabilities of those of skill in the art.

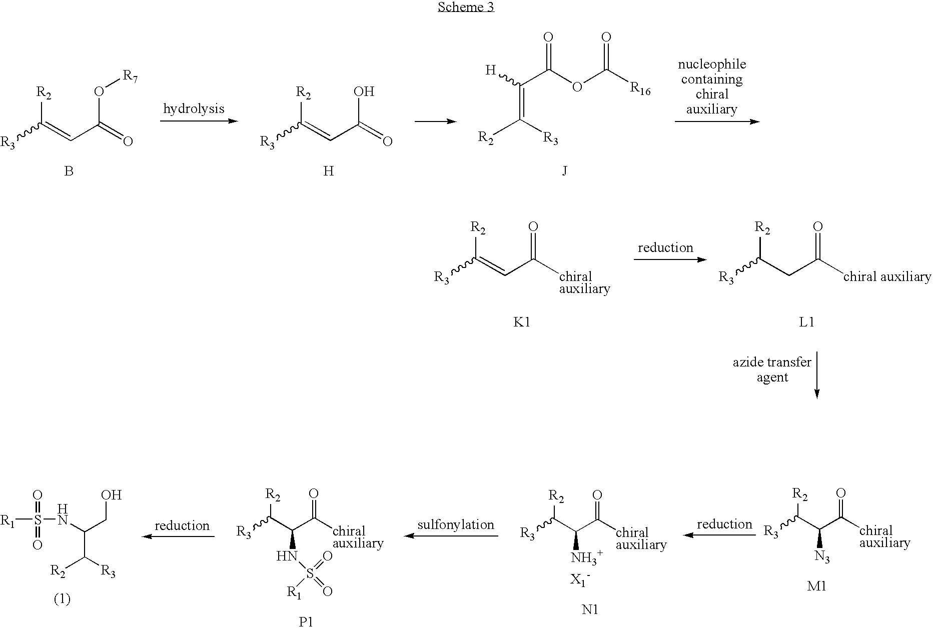

Another example for preparing the compounds of formula (I) is provided in Scheme 4 which includes hydrolysis of ester B1 to afford carboxylic acid H1. The acid is converted to a mixed anhydride via reaction with trimethylacetyl chloride in the presence of triethylamine and THF. The mixed anhydride is then treated with the lithium anion of a chiral, non-racemic, oxazolidinone to afford acyl oxazolidinone K2. Reduction of the olefin moiety of K2 via catalytic hydrogenation affords the reduced oxazolidinone L2. Treatment of L2 with potassium hexamethyldisilazide in THF, followed by reaction with triisopropylbenzenesulfonyl azide, and then an acetic acid quench, yields azide M2 as a mixture of predominately two diastereomers. Reduction of azide M2 as described above yields amine N2 which can be sulfonylated to provide sulfonamide P2. Reduction of the acyl oxazolidinone using lithium borohydride in THF affords compound (I). The mixture of alcohols obtained can be separated most conveniently by chiral, preparative liquid chromatography.

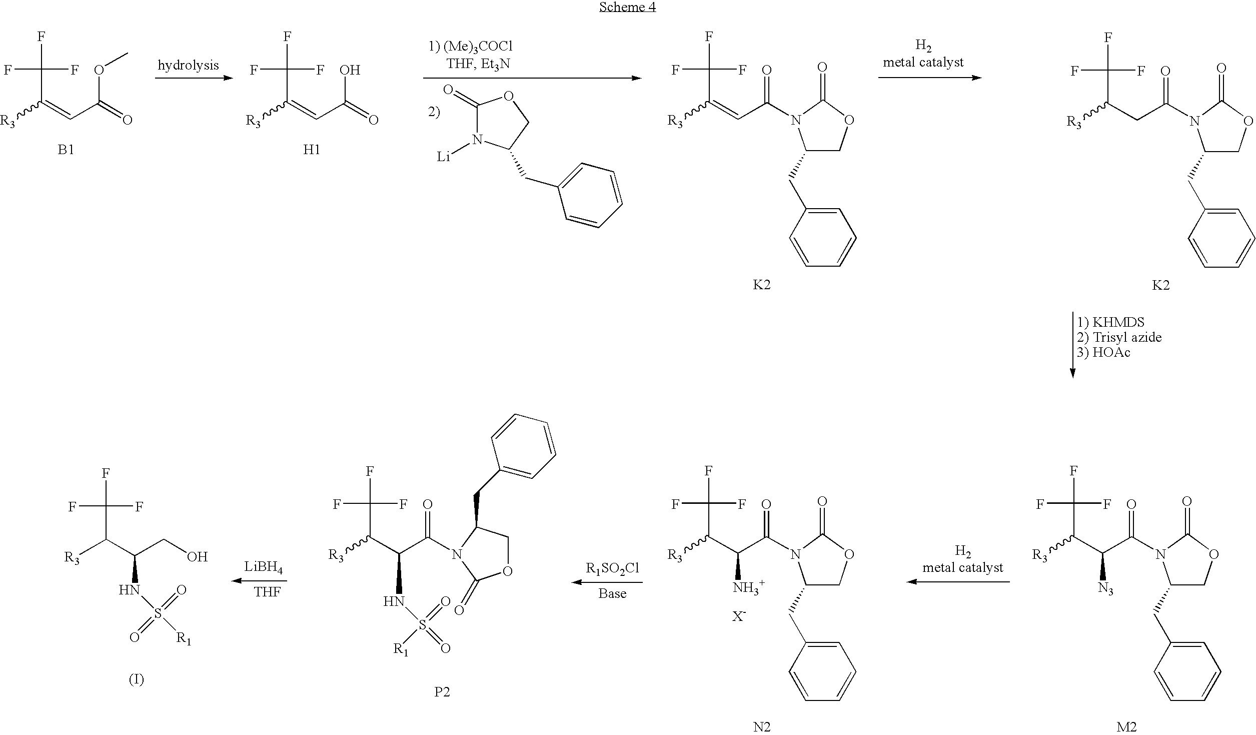

A further route to the compounds of formula (I) is shown in Scheme 5. In this method, a halogenated acetophenone is first converted to α,β-unsaturated carboxylic acid H using techniques known to those of skill in the art. In one embodiment, the α,β-unsaturated carboxylic acid prepared in this step may be compound H1, wherein R₃ is defined above.

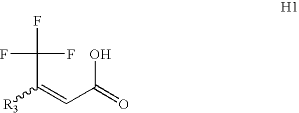

Desirably, the reaction is performed using the reagents and conditions provided in J. R. Johnson in Organic Reactions, 1:210 (1942). In one embodiment, a halogenated acetophenone is converted to the α,β-unsaturated carboxylic acid using sodium acetate and acetic anhydride. In one example, the halogenated acetophenone is compound A. In another example, the halogenated acetophenone is compound A1.

The α, β-unsaturated carboxylic acid is then hydrogenated to a saturated carboxylic acid using techniques in the art including those provided in S. Nishimura in Handbook of Heterogeneous Hydrogenation for Organic Synthesis; Wiley-Interscience: New York, 2001, pages 93-94, which is hereby incorporated by reference. In one embodiment, the hydrogenation is catalytic hydrogenation. Desirably, the catalytic hydrogenation is performed using hydrogen gas and palladium/carbon. In one example, the carboxylic acid is compound R, where R₂ and R₃ are defined above.

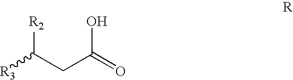

In another example, the carboxylic acid is compound R1, where R₃ is defined above.

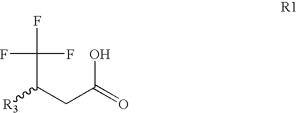

The carboxylic acid is then converted to a mixed anhydride using techniques in the art and described above. The conversion may be performed using the reagents and conditions provided in A. Beckwith cited above and incorporated by reference. Typically, the conversion is performed using R₁₆COX″ as described above, wherein R₁₆ and X″ are defined herein. Desirably, R₁₆COX″ is an acyl chloride. In one example, the acyl chloride is trimethylacetyl chloride, isovaleroyl chloride or diphenylacetyl chloride. The conversion is desirably performed in the presence of a second base as described above. The second base may be selected by one of skill in the art including, without limitation, a tertiary base such as triethylamine. In one example, the mixed anhydride is compound S, wherein R₂, R₃, and R₁₆ are defined above.

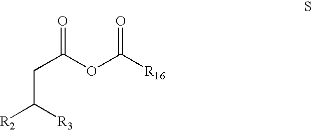

In another example, the mixed anhydride is compound S1, wherein R₃ and R₁₆ are defined above.

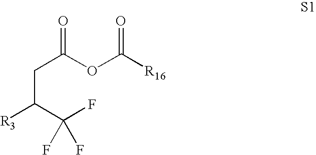

The mixed anhydride is then reacted with a nucleophile containing a chiral auxiliary, as defined above. Desirably, the nucleophile containing a chiral auxiliary is an oxazolidinone or imidazolidinone. In one embodiment, the nucleophile containing a chiral auxiliary is lithium (S)-4-phenyloxazolidin-2-one. In another embodiment, the nucleophile containing a chiral auxiliary is lithium (S)-4-benzyloxazolidin-2-one.

In one example, compound L is prepared from reaction of the mixed anhydride S with the nucleophile containing a chiral auxiliary. In another example, compound L1 is prepared from reaction of the mixed anhydride with the nucleophile containing a chiral auxiliary. In a further example, compound L2 is prepared from reaction of the mixed anhydride with the nucleophile containing a chiral auxiliary.

This compound is then reacted with a base, followed by conversion to an azido-imide using the reagents and conditions discussed above. This conversion is typically performed using an azide transfer agent, including those described in D. A. Evans cited above and incorporated by reference. In one embodiment, the azide transfer agent is triisopropylbenzenesulfonyl azide. In one example, azido-imide M is prepared. In a further example, azido-imide M1 is prepared. In another example, azido-imide M2 is prepared.

The azido-imide is then reduced to an amino-imide salt using techniques known in the art and those specifically described above. In one example, the amino-imide is compound N. In a further example, the amino-imide is compound N1. In another example, the amino-imide is compound N2. In a further example, the amino-imide is compound N3.

The amino-imide is then sulfonylated to a sulfonamido-imide using the techniques and reagents described above. Desirably, the sulfonylation is performed using a sulfonyl chloride or sulfonic anhydride. In one example, the sulfonamido-imide is compound P. In a further example, the sulfonamido-imide is compound P1. In another example, the sulfonamide-imide is compound P2.

The sulfonamido-imide is then reduced to the compound of formula (I) using techniques known to those skilled in the art as discussed above.

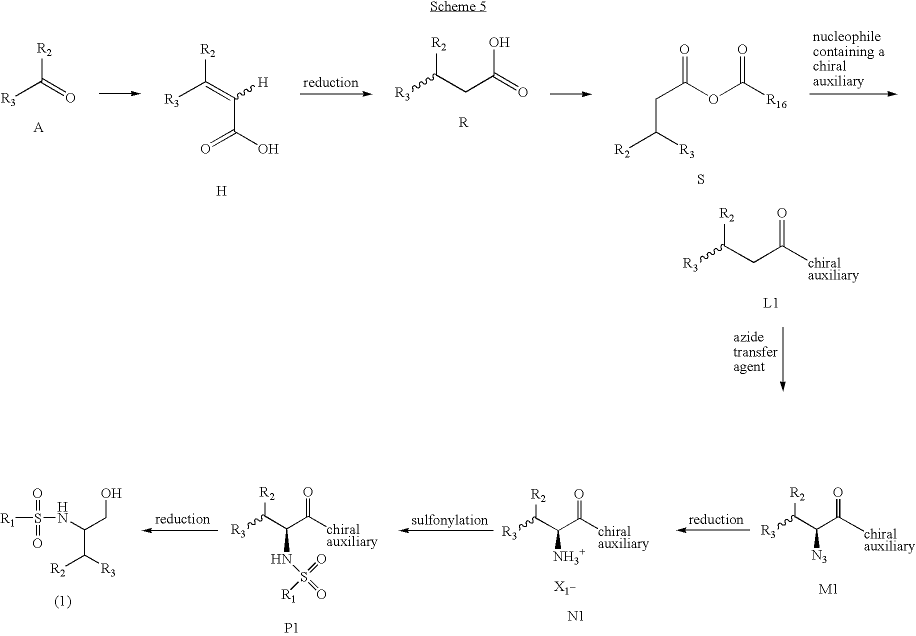

In one example, an alternate route to oxazolidinone L2 and azide M2 is shown in Scheme 6. Trifluoromethylketone A1 is converted to the α,βunsaturated carboxylic acid H1 which is hydrogenated to yield saturated carboxylic acid R1. Conversion of acid R1 to oxazolidinone L2 is performed. The stereoisomers L2 are separated by chromatography and then converted to a single isomer of azide M2.

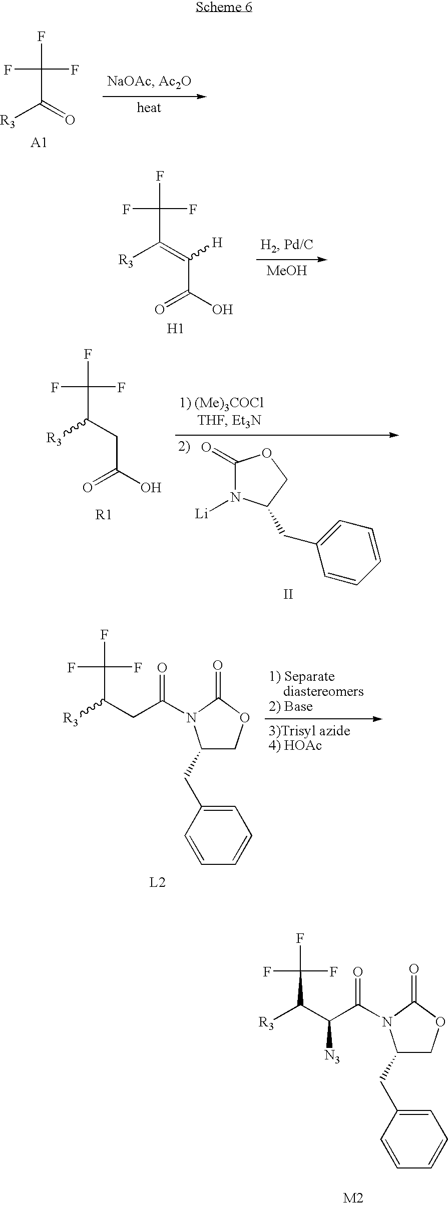

Yet another method for preparing the compounds of formula (I) is described in Scheme 7. In this method, a halogenated acetophenone, an alkyl isocyanoacetate, and a base are reacted using the procedure of Enders, D. et al., Synthesis (2005) pages 306-310, which is incorporated by reference herein. Desirably, the alkyl isocyanoacetate is CNCH₂CO₂R₁₇ and R₁₇ is C₁ to C₆ alkyl or substituted C₁ to C₆ alkyl. In one example, the halogenated acetophenone is compound A. In another example, the halogenated acetophenone is A1.

By doing so, the compound T is prepared, wherein R₂, R₃, and R₁₇ are defined above.

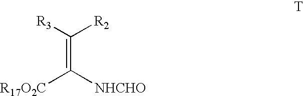

In another example, compound T1 is prepared, wherein R₃ and R₁₇ are defined above.

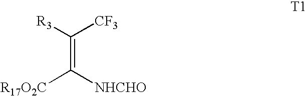

This compound is then reduced using the reagents and conditions provided in R. Larock in Comprehensive Organic Transformations; Wiley-VCH: New York, 1999, pages 20-23 and 1117-1120, which is hereby incorporated by reference. In one embodiment, the reduction is performed using sodium borohydride in methanol and then reacted with lithium borohydride to provide compound U, wherein R₂ and R₃ are defined above.

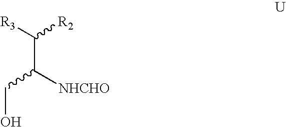

In another example, compound U1 is prepared from the reduction with sodium borohydride and reaction with lithium borohydride.

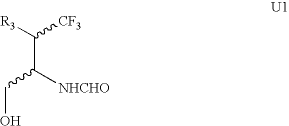

The next step includes hydrolysis with an acid to provide an amine. One of skill in the art would readily be able to select a suitable acid for use in the hydrolysis. In one embodiment, the acid is hydrochloric acid. However, the hydrolysis may be performed by one of skill in the art utilizing the hydrolysis reagents and conditions discussed in A. Beckwith in The Chemistry of Amides, J. Zabicky, Ed., Interscience Publishers: New York, 1970, pages 816-833, which is hereby incorporated by reference. Typically, the amine is present as a racemic mixture of two diastereomers. In one example, amine is compound V, wherein R₂ and R₃ are defined herein.

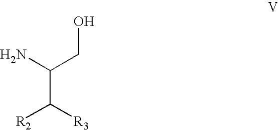

In another example, the amine is compound V1, wherein R₃ is defined herein.

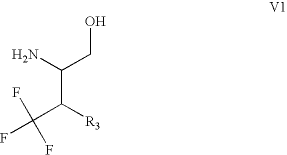

The amine is then sulfonylated using the techniques and reagents described above including, without limitation, sulfonyl chloride or sulfonic anhydride, to provide the compound of formula (I).

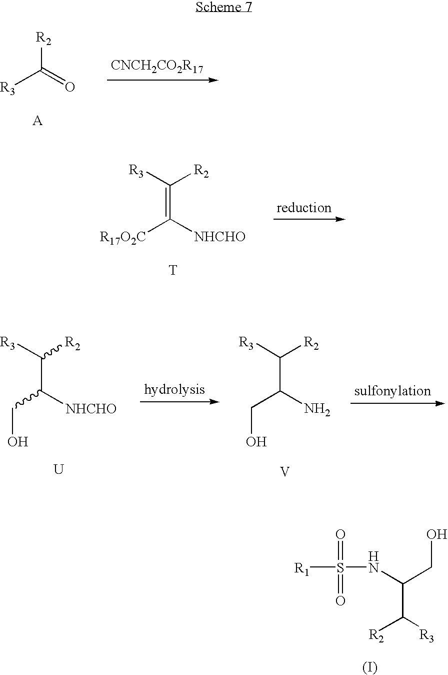

In one example (Scheme 8), the method includes reacting trifluoromethyl acetophenone A1 with an alkyl isocyanoacetate using the method of Enders, D. et al., Synthesis (2005) pages 306-310 to afford tetra-substituted olefin T1. The olefin group of T1 is reduced by treatment with sodium borohydride in methanol. The crude material is then treated with lithium borohydride in THF to produce alcohol U1 as a racemic mixture of two diastereomers (four total isomers). Acid hydrolysis of the formamide group yields the corresponding amine isomers which are sulfonylated to afford compound (I) after chiral, preparatory liquid chromatographic separation.

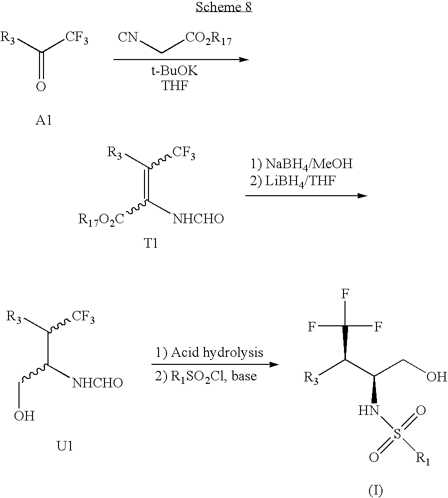

A further method of preparing the compounds of formula I, which method is a variation of the method described in Scheme 3, is provided in Scheme 9. In this method, an α,β-unsaturated ester is hydrolyzed to an α,β-unsaturated carboxylic acid using the reagents and techniques described above. In one example, the α,β-unsaturated ester is compound B. In another example, the α,β-unsaturated ester is compound B1.

Hydrolysis of the α,β-unsaturated ester, which conditions and reagents are discussed and provided above, thereby provides an α,β-unsaturated carboxylic acid. In one example, the α,β-unsaturated carboxylic acid is compound H. In another example, the α,β-unsaturated carboxylic acid is compound H1.

The carboxylic acid is then converted to a mixed anhydride using techniques and reagents known to those of skill in the art as described above and recited in A. Beckwith in The Chemistry of Amides, J. Zabicky, Ed., Interscience Publishers: New York, 1970, page 91 cited above and incorporated by reference. Typically, the conversion is performed using R₁₆COX″, wherein R₁₆ and X″ are defined above. Desirably, R₁₆COX″ is an acyl chloride. In one example, the acyl chloride is trimethylacetyl chloride, isovaleroyl chloride or diphenylacetyl chloride, among others. The conversion is desirably performed in the presence of a second base which may readily be selected by one skilled in the art. In one embodiment, the second base is a tertiary amine base such as triethylamine, among others. In one example, the mixed anhydride is compound J. In another example, mixed anhydride J1 is prepared.

The mixed anhydride is then reacted with a nucleophile that contains a chiral auxiliary as discussed in detail above. In one embodiment, the nucleophile containing a chiral auxiliary is an oxazolidinone. In another embodiment, the nucleophile containing a chiral auxiliary is lithium (S)-4-benzyloxazolidin-2-one.

In one example, compound K is prepared. In a further example, compound K1 is prepared. In another example, compound K2 is prepared.

This compound may then be reduced using techniques known to those of skill in the art including, without limitation, catalytic hydrogenation. Desirably, the hydrogenation is performed using the reagents and conditions provided in Ghosh, A. K. and Liu, W. J. Org. Chem. 60: 6198-6201 (1995), which is hereby incorporated by reference. In one example, the hydrogenation is performed using Pd—C. In another example, the reduction provides compound L. In a further example, the reduction provides compound L1. In yet another example, the reduction provides compound L2.

This compound is then reacted with a base which may be readily selected by one of skill in the art and described in D. A. Evans cited above and incorporated by reference. Suitable bases for use in this step include, without limitation, potassium hexamethyldisilazide. Following reaction with a base, the product is converted to an azido-imide using an azide transfer agent. One of skill in the art would readily be able to select a suitable azide transfer agent including those described in D. A. Evans cited above and incorporated by reference. In one embodiment, the azide transfer agent is triisopropylbenzenesulfonyl azide. In one example, the azido-imide is compound M. In a further example, the azido-imide is compound M1. In another example, the azido-imide is compound M2.

The azido-imide is then reduced to an azido-alcohol by reducing the pendant carbonyl group to provide the azido-alcohol. The reduction is typically performed using a reducing agent which may be readily selected by one skilled in the art. In one example, the reducing agent is lithium borohydride. In one example, the azido-alcohol is compound X, wherein R₂ and R₃ are defined above.

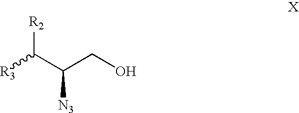

In another example, the azido-alcohol is compound X1, wherein R₃ is defined above.

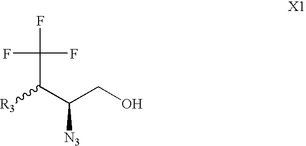

The azido-alcohol is then reduced to an amino-alcohol using techniques known to those of skill in the art. Desirably, the reduction is performed via catalytic hydrogenation using the reagents and conditions provided in S. Nishimura in Handbook of Heterogeneous Hydrogenation for Organic Synthesis; Wiley-Interscience: New York, 2001, pages 377-379, which is hereby incorporated by reference. In one embodiment, the hydrogenation is performed using hydrogen gas and a metal catalyst. In one example, the amino-alcohol is compound Y, wherein R₂ and R₃ are defined above.

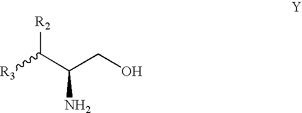

In another example, the amino-alcohol is compound Y1, wherein R₃ is defined above.

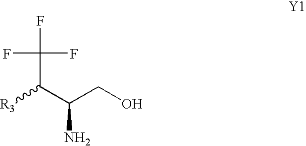

Finally, the amino-alcohol is sulfonylated as described above using techniques and reagents known in the art to provide the compounds of formula (I). Desirably, the amino-alcohol is sulfonylated using a sulfonyl chloride or sulfonic anhydride.

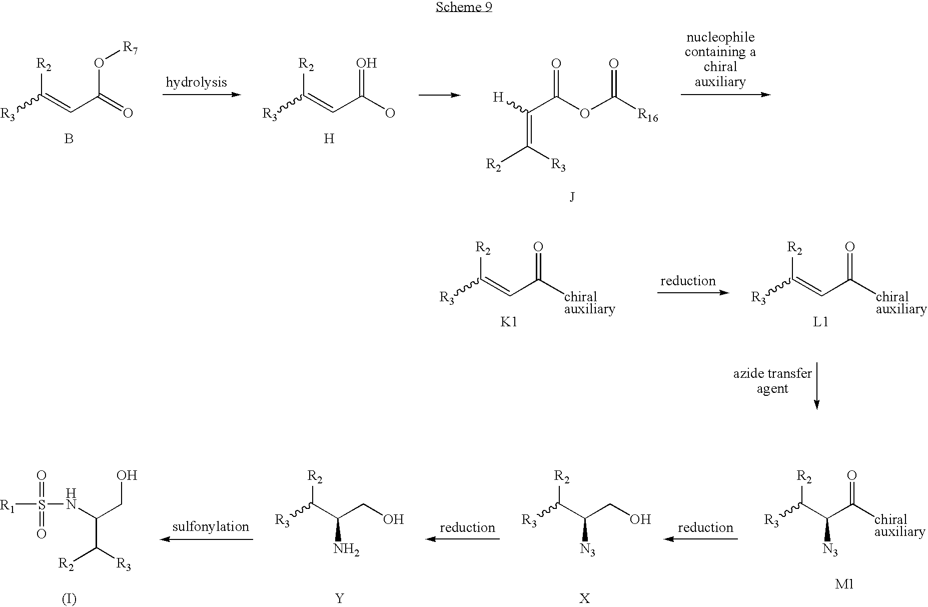

In still another example, one method for preparing the compounds of formula (I) is provided in Scheme 10 and includes reducing the pendant carbonyl of the oxazolidinone group of azide M1 with lithium borohydride to afford azide alcohol X1. Reduction of the azide group of X1 affords amino alcohol Y1. Amino alcohol Y1 can then be either directly converted to sulfonamide compound (I) or a suitable hydroxyl protecting group known to those skilled in the art can be employed prior to sulfonylation of the amine. Compound (I) can then be isolated (either directly or after removal of a suitable hydroxyl protecting group) by chiral, preparative liquid chromatographic separation.

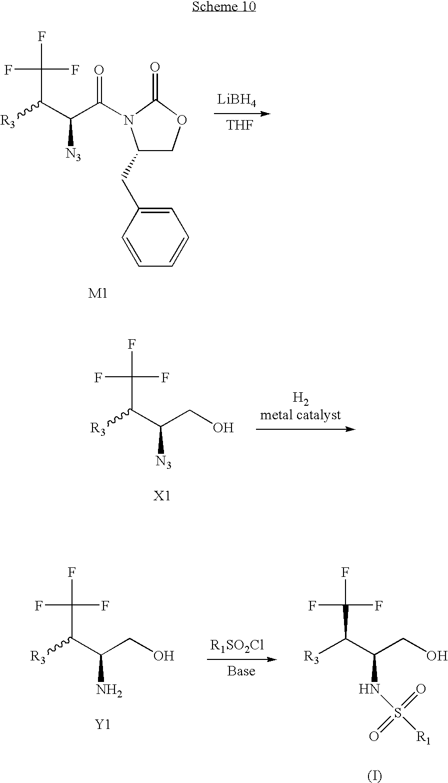

### Analysis of Compounds Prepared using the Described Methods

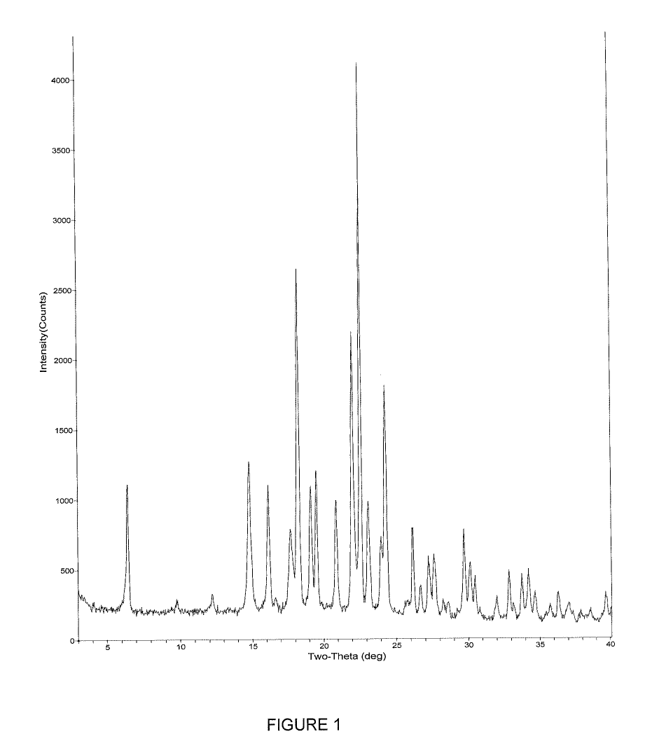

The powder XRD pattern of 5-Chloro-N-[(1S,2S)-2-(3,5-difluorophenyl)-3,3,3-trifluoro-1-(hydroxymethyl)propyl]thiophene-2-sulfonamide described herein was obtained using X-ray crystallographic techniques known to those of skill in the art. See, FIG. 1. In one embodiment, the XRD pattern of 5-Chloro-N-[(1S,2S)-2-(3,5-difluorophenyl)-3,3,3-trifluoro-1-(hydroxymethyl)propyl]thiophene-2-sulfonamide contains one large peak and several smaller peaks. The XRD for 5-Chloro-N-[(1S,2S)-2-(3,5-difluorophenyl)-3,3,3-trifluoro-1-(hydroxymethyl)propyl]thiophene-2-sulfonamide includes a peak at 2θ of about 6.4°±0.3°. The XRD for 5-Chloro-N-[(1S,2S)-2-(3,5-difluorophenyl)-3,3,3-trifluoro-1-(hydroxymethyl)propyl]thiophene-2-sulfonamide may also include one or more peaks at 2θ of about 14.8°±0.3°, 16.1°±0.3°, 18.3°±0.3°, 19.1°±0.3°, 19.5°±0.3°, 22.1°±0.3°, 22.6°±0.3°, or 24.3°±0.3° at varying intensities. One of skill in the art would readily recognize that the intensities of the peaks of the powder X-ray diffraction pattern may vary. In one embodiment, the intensities of one or more peaks of the powder X-ray diffraction pattern may vary due to crystal shape, crystal size, among others.

### Methods of Using the Compounds

Compounds of formula (1) are inhibitors of beta amyloid production. In preliminary studies using protease specific assays, exemplary compounds of formula (I) have been shown to exhibit specific inhibition with respect to protease activity. Thus, the compounds are useful for treatment and prevention of a variety of conditions in which modulation of beta amyloid levels provides a therapeutic benefit. Such conditions include, e.g., amyloid angiopathy, cerebral amyloid angiopathy, systemic amyloidosis, Alzheimer's Disease (AD), hereditary cerebral hemorrhage with amyloidosis of the Dutch type, inclusion body myositis, Down's syndrome, mild cognitive impairment (MCI), among others. Desirably, the compounds are administered in an amount sufficient to alleviate the symptoms or progress of the condition.

In addition, the compounds of formula (I) may be utilized in generating reagents useful in diagnosis of conditions associated with abnormal levels of beta amyloid. For example, the compounds of formula (I) may be used to generate antibodies, which would be useful in a variety of diagnostic assays. Methods for generating monoclonal, polyclonal, recombinant, and synthetic antibodies or fragments thereof, are well known to those of skill in the art. See, e.g., E. Mark and Padlin, “Humanization of Monoclonal Antibodies”, Chapter 4, The Handbook of Experimental Pharmacology, Vol. 113, The Pharmacology of Monoclonal Antibodies, Springer-Verlag (June, 1994); Kohler and Milstein and the many known modifications thereof; International Patent Publication No. WO86/01533; British Patent Application Publication No. GB2188638A; Amit et al., Science, 233:747-753 (1986); Queen et al., Proc. Nat'l. Acad. Sci. USA, 86:10029-10033 (1989); International Patent Publication No. WO90/07861; Riechmann et al., Nature, 332:323-327 (1988); and Huse et al., Science, 246:1275-1281 (1988), which are hereby incorporated by reference. Alternatively, the compounds of formula (I) may themselves be used in such diagnostic assays. Regardless of the reagent selected (e.g., antibody or compound of formula (I)), suitable diagnostic formats including, e.g., radioimmunoassays and enzyme-linked immunosorbent assays (ELISAs), are well known to those of skill in the art and are not a limitation.

Additionally, cellular, cell-free and in vivo screening methods to detect inhibitors of beta amyloid production are known in the art. Such assays may include radioimmunoassays and enzyme-linked immunosorbent assay (ELISA), among others. See, e.g., P. D. Mehta, et al., Techniques in Diagnostic Pathology, vol. 2, eds., Bullock et al., Academic Press, Boston, pages 99-112 (1991), International Patent Publication No. WO98/22493, European Patent No. 0652009, U.S. Pat. Nos. 5,703,129 and 5,593,846, which are hereby incorporated by reference. Selection of an appropriate in vitro or in vivo screening assay is not a limitation.

In one embodiment, methods of inhibiting beta amyloid production in a subject are provided and include delivering a compound of formula (I) or a pharmaceutical composition containing a compound of formula (I) to the subject.

### Pharmaceutical Compositions and Formulations

Also provided are pharmaceutical compositions which contain one or more compounds of formula I, a prodrug of the compound of formula I, or combinations thereof.

The compounds described herein may be administered to a subject by any desirable route, taking into consideration the specific condition for which it has been selected. By “subject” is meant any suitable human which have been recognized as having or at risk of having one or more of the conditions for which modulation of beta amyloid levels is desirable. Thus, the compounds of formula (I) are useful for treatment and/or prevention of a number of human conditions. As used herein, “prevention” encompasses prevention of symptoms in a subject who has been identified as at risk for the condition, but has not yet been diagnosed with the same and/or who has not yet presented any symptoms thereof.

These compounds may be delivered or administered by any suitable route of delivery, e.g., oral, injection, inhalation (including oral, intranasal and intratracheal), transdermal, intravenous, subcutaneous, intramuscular, sublingual, intracranial, epidural, intratracheal, rectal, vaginal, among others. Most desirably, the compounds are delivered orally, by inhalation or by a suitable parenteral route. As known in the art, rectal and vaginal delivery may be via a suppository. The compounds may be formulated in combination with conventional pharmaceutical carriers that are physiologically compatible. Optionally, one or more of the compounds of formula (I) may be mixed with other active agents.

Suitable physiologically compatible carriers may be readily selected by one of skill in the art. For example, suitable solid carriers include, among others, one or more substances which may also act as flavoring agents, lubricants, solubilizers, suspending agents, fillers, glidants, compression aids, binders or tablet-disintegrating agents or encapsulating materials. In powders, the carrier is a finely divided solid, which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, e.g., calcium or dicalcium phosphate, magnesium stearate, talc, starch, sugars (including, e.g., lactose and sucrose), cellulose (including, e.g., microcrystalline cellulose, methyl cellulose, sodium caroboxymethyl cellulose), polyvinylpyrrolidine, low melting waxes, ion exchange resins, and kaolin.

Liquid carriers may be used in preparing solutions, suspensions, emulsions, syrups and elixirs. The active ingredient(s) can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, suspending agents, thickening agents, viscosity regulators, stabilizers or osmo-regulators. Suitable examples of liquid carriers for oral and parenteral administration include water (particularly containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil, arachis oil, corn oil, peanut oil, and sesame oil). For parenteral administration, the carrier can also be an oily ester such as ethyl oleate and isopropyl myristate. Sterile liquid carriers are used in sterile liquid to form compositions for parenteral administration.

Optionally, additives customarily employed in the preparation of pharmaceutical compositions may be included in the compositions. Such components include, e.g., sweeteners or other flavoring agents, coloring agents, preservatives, and antioxidants, e.g., vitamin E, ascorbic acid, butylated hydroxytoluene (BHT) and butylated hydroxyanisole (BHA).

Liquid pharmaceutical compositions that are sterile solutions or suspensions can be utilized by, e.g., intramuscular, intraperitoneal or subcutaneous injection. Sterile solutions can also be administered intravenously. Oral administration may be either in liquid or solid composition form.

Suitably, when prepared for use as an inhalant, the pharmaceutical compositions are prepared as fluid unit doses using a compound of formula (I) and a suitable pharmaceutical vehicle for delivery by an atomizing spray pump, or by dry powder for insufflation. For use as aerosols, the compound is formulated for and packaged in a pressurized aerosol container together with a gaseous or liquefied propellant, e.g., dichlorodifluoromethane, carbon dioxide, nitrogen, propane, and the like, with the usual components such as cosolvents and wetting agents, as may be necessary or desirable. For example, also provided is the delivery of a metered dose for oral or intranasal inhalation in one, two, or more actuations. Suitably, a dose is delivered in one or two actuations. However, other suitable delivery methods may be readily determined.

Preferably, the pharmaceutical composition is in unit dosage form, e.g., as tablets or capsules. In such form, the composition is sub-divided in unit dose containing appropriate quantities of the active ingredient; the unit dosage forms can be packaged compositions, e.g., packeted powders, vials, ampoules, prefilled syringes or sachets containing liquids. The unit dosage form can be, e.g., a capsule or tablet itself, or it can be the appropriate number of any such compositions in package form.

As described herein, a therapeutically or prophylactically useful amount of a compound of formula (I) is that amount of a compound which alleviates the symptoms of the disease, e.g., AD, or which prevents the onset of symptoms, or the onset of more severe symptoms. The useful amounts of a compound may vary depending upon the formulation and route of delivery. For example, higher amounts may be delivered orally than when the compound is formulated for injection or inhalation, in order to deliver a biologically equivalent amount of the drug. Suitably, an individual dose (i.e., per unit) of a compound is in the range from about 1 μg/kg to about 10 g/kg. However, in certain embodiments, these doses may be selected from a lower range, e.g., from about 1 μg/kg to about 200 mg/kg, more preferably 10 μg/kg to about 10 mg/kg, and most preferably about 100 μg/kg to about 1 mg/kg. Desirably, these amounts are provided on a daily basis. However, the dosage to be used in the treatment or prevention of a specific cognitive deficit or other condition may be subjectively determined by the attending physician. The variables involved include the specific cognitive deficit and the size, age and response pattern of the patient. For example, based upon the activity profile and potency of the compounds described herein, a starting dose of about 130 to about 300 mg per day with gradual increases in the daily dose to about 1000 mg per day may provide the desired dosage level in the human.

Alternatively, the use of sustained delivery devices may be desirable in order to avoid the necessity for the patient to take medications on a daily basis. “Sustained delivery” is defined as delaying the release of an active agent, i.e., a compound of formula I, until after placement in a delivery environment, followed by a sustained release of the agent at a later time. Those of skill in the art know suitable sustained delivery devices. Examples of suitable sustained delivery devices include, e.g., hydrogels (U.S. Pat. Nos. 5,266,325; 4,959,217; and 5,292,515), an osmotic pump, such as described by Alza (U.S. Pat. Nos. 4,295,987 and 5,273,752) or Merck (European Patent No. 314,206), among others; hydrophobic membrane materials, such as ethylenemethacrylate (EMA) and ethylenevinylacetate (EVA); bioresorbable polymer systems (see, e.g., International Patent Publication No. WO 98/44964, Bioxid and Cellomeda; U.S. Pat. No. 5,756,127 and U.S. Pat. No. 5,854,388); other bioresorbable implant devices have been described as being composed of, e.g., polyesters, polyanhydrides, or lactic acid/glycolic acid copolymers (see, e.g., U.S. Pat. No. 5,817,343 (Alkermes Inc.)), all of which documents which are hereby incorporated by references. For use in such sustained delivery devices, the compounds may be formulated as described herein.

In another aspect, pharmaceutical kits for delivery of a product are provided. Suitably, the kit contains packaging or a container with the compound formulated for the desired delivery route. For example, if the kit is designed for administration by inhalation, it may contain a suspension containing a compound of formula (I) formulated for aerosol or spray delivery of a predetermined dose by inhalation. Suitably, the kit contains instructions on dosing and an insert regarding the active agent. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of the spray pump or other delivery device.

Other suitable components to such kits will be readily apparent to one of skill in the art, taking into consideration the desired indication and the delivery route. The doses may be repeated daily, weekly, or monthly, for a predetermined length of time or as prescribed.

The following examples are illustrative of compounds of formula (I) and methods of synthesizing same. It will be readily understood by one of skill in the art that the specific conditions described herein for producing these compounds can be varied without departing from the scope of the present invention. It will be further understood that other compounds of formula I, as well as other salts, hydrates, and/or prodrugs thereof, are within the scope of the invention.

### EXAMPLES

### Examples 1-5

### 5-Chloro-N-[3,3,3-trifluoro-1-(hydroxymethyl)-2-phenylpropyl]thiophene-2-sulfonamide (1)

### 5-Chloro-N-[(1R, 2S)-3,3,3-trifluoro-1-(hydroxymethyl)-2-phenylpropyl]thiophene-2-sulfonamide (3)

### 5-Chloro-N-[(1S, 2R)-3,3,3-trifluoro-1-(hydroxymethyl)-2-phenylpropyl]thiophene-2-sulfonamide (4)

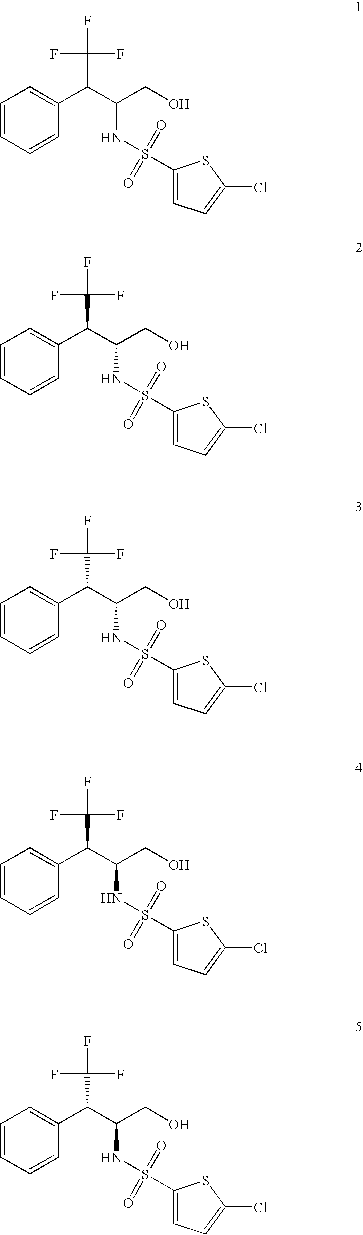

### Step 1: Methyl-4,4,4-trifluoro-3-phenylbut-2-enoate

To a suspension of NaH (3.29 g, 82.2 mmol, 60% dispersion in mineral oil) in dry THF (400 mL) and cooled to 0° C., was added dropwise a solution of trimethyl phosphonoacetate (15.0 g, 13.3 mL, 82.2 mmol) in THF (25 mL). The reaction mixture was stirred for 0.5 hours at 0° C. after which the cooling bath was removed. The reaction was allowed to stir for 1 hour at room temperature, then trifluoroacetophenone (13.06 g, 75 mmol, 10.5 mL) was added slowly. The reaction mixture was allowed to stir for 4 hours at room temperature, poured into saturated sodium bicarbonate and the aqueous mixture was partitioned with EtOAc. The organic layer was separated and the aqueous layer was extracted with EtOAc. The combined organic layers were washed with brine, dried over Na₂SO₄, filtered and concentrated in vacuo to provide 18.15 g of an oil as a mixture of E/Z isomers contaminated with 6 mol % of the starting phosphonate. This material was used as is in the next step. ¹H NMR 500 MHz (dimethylsulfoxide (DMSO)-d₆): δ 3.50 (s, 3 H), 6.84 (d, 0.8 H, J=1.16 Hz), 6.90 (s, 0.2 H), 7.23-7.25 (m, 1 H), 7.39-7.46 (m, 4 H).

### Step 2: Methyl 4,4,4-trifluoro-3-phenylbutanoate

A solution of the ester from Step 1 (2.3 g, 10 mmol) in MeOH (200 mL) was hydrogenated over 20% Pd(OH)₂/C (500 mg) at 1 atm hydrogen for 1 hour. The reaction mixture was filtered through the CELITE® reagent. The filtrate was concentrated in vacuo to yield a gray oil. Flash chromatography on SiO₂ EtOAc/hexanes (5/95) provided 1.89 g of the title compound (81%) as a colorless oil. ¹H NMR 500 MHz (DMSO-d₆): δ 2.96-3.08 (m, 2 H), 3.49 (s, 1 H), 4.00-4.06 (m, 1 H), 7.30-7.39 (m, 5 H). MS (+ESI): m/z 233 [M+H]⁺.

### Step 3: Methyl-2-azido-4,4,4-trifluoro-3-phenylbutanoate

A solution, cooled to −78° C., of lithium diisopropylamide (LDA) (3.83 mL, 6.9 mmol, 1.8 M heptane/THF/ethylbenzene) in THF (10 mL) was added, via cannula, to a precooled (−78° C.) solution of methyl 4,4,4-trifluoro-3-phenylbutanoate (1.4 g, 6.0 mmol) in THF (9 mL). The reaction mixture was stirred at −78° C. for 0.5 hours after which a −78° C. solution of trisyl azide (2.54 g, 8.22 mmol) in THF (9 mL) was added rapidly via cannula. After 3 minutes, AcOH (2.53 mmol, 152 mg, 139 μL) was added to the reaction mixture. The cooling bath was removed and the reaction mixture was allowed to stir at room temperature overnight. The reaction mixture was diluted with saturated NaCl and the mixture was stirred for 30 seconds. The mixture was extracted with CH₂Cl₂. The combined organic layers were dried over Na₂SO₄, filtered, and concentrated in vacuo to provide a yellow oil. Flash chromatography on SiO₂ EtOAc/hexanes (5/95) provided the title compound (569 mg, 35%) as a 9:1 mixture of diastereomers. ¹H NMR 500 MHz (DMSO-d₆): δ 3.54 (s, 0.3 H), 3.64 (s, 2.7 H), 4.29-4.33 (m, 1 H), 4.91 (d, J=7.54 Hz, 0.9 H), 5.22 (d, J=5.80 Hz, 0.1 H), 7.33-7.37 (m, 3 H), 7.43-7.45 (m, 2 H).

### Step 4: 2-Amino-4,4,4,-trifluoro-3-phenyl-butyric acid methyl ester

A solution of methyl-2-azido-4,4,4-trifluoro-3-phenylbutanoate (516 mg, 1.89 mmol) in EtOAc (15 mL) was hydrogenated at 1 atm over 10% Pd/C (125 mg). After 1.5 hours the reaction mixture was filtered through the CELITE® reagent. The filtrate was concentrated in vacuo to give 400 mg of a colorless to light yellow oil. This material was purified by flash chromatography on SiO₂ (gradient EtOAc/hexanes) to yield the title compound as a colorless oil. ¹H NMR 500 MHz (DMSO-d₆): δ 1.20 (s, 0.2 H), 1.72 (s, 1.8 H), 3.37 (s, 0.3 H), 3.59 (s, 2.7 H), 3.84-3.89 (m, 1 H), 4.00 (m, 1 H), 7.30-7.38 (m, 5 H); MS (+ESI): m/z 248 [M+H]⁺.

### Step 5: 2-(5-Chloro-thiophene-2-sulfonylamino)-4,4,4,-trifluoro-3-phenyl-butyric acid methyl ester

To a cooled (0° C.) solution of 2-amino-4,4,4,-trifluoro-3-phenyl-butyric acid methyl ester (200 mg, 0.81 mmol) from Step 4 in dichloromethane (DCM; 5 mL) was added 5-chloro-2-thienylsulfonyl chloride (193 mg, 0.89 mmol). Pyridine (1.5 eq., 1.21 mmol, 96 mg, 109 μL) was then added to the reaction mixture. The reaction mixture was allowed to warm to room temperature overnight. The reaction mixture was diluted with CH₂Cl₂ and washed with water. The organic layer was dried over Na₂SO₄, filtered, and concentrated in vacuo to give a yellow oil. Flash chromatography on SiO₂ (gradient EtOAc/hexanes) yielded the title compound (268 mg, 77%) as an 8.4:1 mixture of diastereomers. ¹H NMR 500 MHz (DMSO-d₆): δ 3.06 (s, 0.36 H), 3.50 (s, 2.64 H), 3.93-3.98 (m, 1 H), 4.40-4.45 (m, 1 H), 7.10 (d, J=4.03 Hz, 0.88 H), 7.17 (d, J=4.15 Hz, 0.12 H), 7.24-7.34 (m, 5.88 H), 7.38 (d, J=4.15 Hz, 0.12 H), 9.02 (d, J=9.15 Hz, 0.88 H), 9.36 (d, J=9.15 Hz, 0.12 H). MS (−ESI): m/z 426 [M−H]⁻.

### Step 6: 5-Chloro-N-[3,3,3-trifluoro-1-(hydroxymethyl)-2-phenylpropyl]thiophene-2-sulfonamide (1)

To a solution of 2-(5-chloro-thiophene-2-sulfonylamino)-4,4,4,-trifluoro-3-phenyl-butyric acid methyl ester (161 mg, 0.377 mmol) in dry THF (1.5 mL) was added LiBH₄ (2.0 M in THF, 0.754 mmol, 377 μL). The reaction mixture was stirred at room temperature overnight. 2N HCl was added carefully until no more foam appeared. The solvent was removed in vacuo and water (2 mL) was added to the residue. The solution was extracted with EtOAc. The combined organic extracts were dried over Na₂SO₄, filtered and concentrated in vacuo to give a white solid. Flash chromatography on SiO₂ (gradient EtOAc/hexanes) yielded the title compound (116 mg, 77%) as a 9:1 mixture of diastereomers. ¹H NMR 500 MHz (DMSO-d₆): δ 2.86-2.97 (m, 2 H), 3.79-3.87 (m, 2 H), 4.66 (t, J=9.64 Hz, 0.1 H), 5.07 (t, J=2.92 Hz, 0.9 H), 7.15 (d, J=2.32 Hz, 1 H), 7.28-7.37 (m, 5.9 H), 7.46 (d, J=4.02 Hz, 0.1 H), 7.80 (d, J=9.03 Hz, 0.9 H), 8.35 (d, J=8.81 Hz, 0.1 H). Corrected ¹H NMR 500 MHz (DMSO-d₆): δ 2.86-2.97 (m, 2 H), 3.79-3.87 (m, 2 H), 5.07 (t, J=2.92 Hz, 1 H), 7.15 (d, J=2.32 Hz, 1 H), 7.28-7.37 (m, 6 H), 7.80 (d, J=9.03 Hz, 1 H). MS (−ESI): m/z 398 [M−H]⁻.

### Step 7: 5-Chloro-N-[(1R,2S)-3,3,3-trifluoro-1-(hydroxymethyl)-2-phenylpropyl]thiophene-2-sulfonamide (3)

### Preparation of 5-Chloro-N-[(1S,2R)-3,3,3-trifluoro-1-(hydroxymethyl)-2-phenylpropyl]thiophene-2-sulfonamide (4)

A portion of 5-chloro-N-[3,3,3-trifluoro-1-(hydroxymethyl)-2-phenylpropyl]thiophene-2-sulfonamide from Step 6 was recrystallized from hexanes/Et₂O to yield 5-chloro-N-[(1R*,2R*)-3,3,3-trifluoro-1-(hydroxymethyl)-2-phenylpropyl]thiophene-2-sulfonamide and 5-chloro-N-[(1S*,2S*)-3,3,3-trifluoro-1-(hydroxymethyl)-2-phenylpropyl]thiophene-2-sulfonamide as the major diastereomers (these were not purified further). The mother liquors were evaporated and the material was recrystallized from hexanes/Et₂O. This material was purified by flash chromatography on SiO₂ EtOAc/hexanes (25/75) to provide 5-chloro-N-[(1R*,2S*)-3,3,3-trifluoro-1-(hydroxymethyl)-2-phenylpropyl]thiophene-2-sulfonamide and 5-chloro-N-[(1S,2R)-3,3,3-trifluoro-1-(hydroxymethyl)-2-phenylpropyl]thiophene-2-sulfonamide as the minor diastereomers, as an off-white solid containing 5% of the major diastereomer. This sample was subjected to chiral high performance liquid chromatograph (HPLC; the CHIRALCEL® AD column, 2×25 cm, 70% EtOH in hexane) to provide the title compound, 5-chloro-N-[(1S,2R)-3,3,3-trifluoro-1-(hydroxymethyl)-2-phenylpropyl]thiophene-2-sulfonamide (4), as a white solid, and 5-chloro-N-[(1R,2S)-3,3,3-trifluoro-1-(hydroxymethyl)-2-phenylpropyl]thiophene-2-sulfonamide (3).

(3) MS (−ESI): m/z 398 [M−H]⁻.

(4) MS (−ESI): m/z 398 [M−H]⁻.

### Preparation of 5-Chloro-N-[(1R* ,2R*)-3,3,3-trifluoro-1-(hydroxymethyl)-2-phenylpropyl]thiophene-2-sulfonamide (2) and 5-Chloro-N-[(1S*,2S*)-3,3,3-trifluoro-1-hydroxymethyl-2-phenylpropyl]thiophene-2-sulfonamide (5)

The title compounds were prepared using the method of Example 8 described below, Method B, steps 1-3 and using 2,2,2-trifluoroacetophenone instead of 1-(3,5-difluoro-phenyl)-2,2,2-trifluoroethanone. The product was isolated as the racemate and separated into its enantiomers via chiral prep HPLC.

(2) MS (−ESI): m/z 398 [M−H]⁻.

(5) MS (−ESI): m/z 397.9 [M−H]⁻.
CHIRALPREP® LC Conditions: VARIAN® Prep LC; the CHIRALPAK® AD-H column (25×4.6 mm); Mobile phase 15% ethanol in hexanes; Flow rate 1.0 mL/min. Retention times: (2) 6.783 min (50.14% area); (5) 7.577 min (47.52% area).
   * Absolute stereochemistry assigned arbitrarily for these isomers.

Absolute stereochemistry for 5-chloro-N-[(1S,2R)-3,3,3-trifluoro-1-(hydroxymethyl)-2-phenylpropyl]thiophene-2-sulfonamide (4) was assigned from a single crystal X-ray analysis of the material. The absolute stereochemistry of 5-chloro-N-[(1R,2S)-3,3,3-trifluoro-1-(hydroxymethyl)-2-phenylpropyl]thiophene-2-sulfonamide (3) was derived from 4 since the two materials were enantiomers.

### Example 6

### 4-Chloro-N-[(1S,2R)-3,3,3-trifluoro-1-(hydroxymethyl)-2-phenylpropyl]benzenesulfonamide (6)

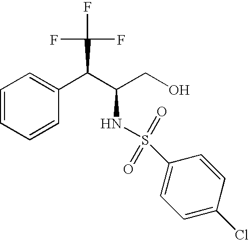

### Step 1: 4,4,4-Trifluoro-3-phenyl-but-2-enoic acid

4,4,4-Trifluoro-3-phenylbut-2-enoic acid was prepared by the method of Sevenard (Tetrahedron Letters, 44, 2003, 7119) from 2,2,2-trifluoro-1-phenylethanone, acetic anhydride and sodium acetate as a 5:1 mixture of E:Z olefins.

### Step 2: 4,4,4-Trifluoro-3-phenylbutanoic acid

A solution of 4,4,4-trifluoro-3-phenylbut-2-enoic acid (6.05 g, 28 mmol) was dissolved in ethanol (200 mL) and was hydrogenated at 1 atm over 10% Pd/C (0.5 g). After 24 hours the solution was filtered to yield the title compound (6 g) as a solid. ¹H NMR (CDCl₃): δ 8.64 (brs, 1 H), 3.86, (m, 1 H), 3.04, (dd, 1 H, J=11, 4.9 Hz), 2.90 (dd, 1 H, J=11, 7.6 Hz).

### Step 3: (4S)-4-Benzyl-(3S)-3-(4,4,4-trifluoro-3-phenylbutanoyl)oxazolidin-2-one

A solution of 4,4,4-trifluoro-3-phenylbutanoic acid (1 g, 6.4 mmol) in THF (30 mL) was cooled to 0° C. under nitrogen, and to this was added triethylamine (0.95 mL, 6.7 mmol) followed by pivaloyl chloride (0.76 mL, 6.5 mmol). The mixture was stirred for 2 hours and then cooled to −78° C.

A solution of (S)-4-benzyloxazolidin-2-one (1.13 g, 6.3 mmol) in THF (20 mL) was cooled to −78° C. under nitrogen. To this solution was added n-BuLi (4 mL, 1.6 M in hexane). The anion was stirred for 30 minutes and was added via cannula to the solution of mixed anhydride described above. The solution was stirred for 30 minutes and then was allowed to warm to room temperature. The reaction was quenched with 1M NaHSO₄ (5 mL). The solvents were removed in vacuo and the residue was partitioned between EtOAc and saturated aqueous sodium bicarbonate solution. The organic layer was removed and the aqueous layer was washed with EtOAc. The organic layers were pooled, extracted once with saturated brine solution, and dried over MgSO₄. Removal of the solvent provided 2.5 g of an oil. Chromatography over silica gel using a gradient elution of EtOAc/hexane provided 520 mg of a single diastereomer (Peak One) and 720 mg of a mixture of diastereomers (36:64). Data for Peak One: ¹H NMR (CDCl₃): δ 7.30 (m, 8 H), 7.14 (d, 2 H, J=6.6 Hz), 4.50 (m, 1 H), 3.73 (dd, 1 H, J=9.7, 20 Hz), 4.10 (m, 3 H), 3.55 (dd, 1 H, J=5.0, 20 Hz), 3.20 (dd, 1 H, J=3.4, 14 Hz), 2.68 (dd, 1 H, J=9.7, 14 Hz).

### Step 4: (S)-3-((2S,3R)-2-Azido-4,4,4-trifluoro-3-phenyl-butyryl)-4-benzyl-oxazolidin-2-one

A solution of oxazolidinone obtained above in Step 3 (Peak One, 0.520 g, 1.3 mmol) in THF (5 mL) was cooled to −78° C. under nitrogen. To this was added a solution (0.5 M in toluene) of potassium hexamethyldisilylamide (3.4 mL, 1.7 mmol). The reaction was stirred at −78° C. for 1 hour. A solution of trisyl azide (0.385 g, 1.93 mmol) in THF (6 mL) was prepared and cooled to −78° C. This solution was added to the anion solution via cannula. After 3 minutes, acetic acid (0.45 mL) was added and the reaction was stirred overnight and allowed to warm to room temperature. The solvents were removed in vacuo and the reaction was diluted with CH₂Cl₂ (40 mL) and saturated brine (40 mL). The layers were separated and the aqueous layer was extracted with CH₂Cl₂. The organic layers were pooled, dried over MgSO₄, filtered and evaporated in vacuo to yield 860 mg of an oil. Chromatography over silica gel using a gradient elution of EtOAc/hexane provided 331 mg of the title compound as an oil. This material was used as was in the next step. ¹H NMR (CDCl₃): δ 5.85 (d, 1 H, J=10.9 Hz).

### Step 5: (S)-3-((2S, 3R)-2-Amino-4,4,4-trifluoro-3-phenyl-butyryl)-4-benzyl-oxazolidin-2-one hydrochloride

A solution of the azide described in Step 4 (0.330 g, 0.7 mmol) dissolved in EtOH (30 mL) containing 1N HCl in diethyl ether (2 mL) was hydrogenated over 10% Pd/C at 1 atmosphere overnight. Filtration of the sample and removal of the solvent yielded 265 mg of the title compound. ¹H NMR (CDCl₃): δ 5.72 (d, 1 H, J=8.6 Hz).

### Step 6: N—(S)-[1-((S)-4-Benzyl-2-oxo-oxazolidine-3-carbonyl)-(R)-3,3,3-trifluoro-2-phenyl-propyl]-4-chlorobenzenesulfonamide

A solution of (S)-3-((2S,3R)-2-Amino-4,4,4-trifluoro-3-phenyl-butyryl)-4-benzyl-oxazolidin-2-one hydrochloride (265 mg, 0.61 mmol) in CH₂Cl₂ (20 mL) was cooled to 0° C. A solution of 4-chloro-phenylsulfonyl chloride (0.193 g, 0.9 mmol) in CH₂Cl₂ (2 mL) was treated with dimethylaminopyridine (DMAP, 151 mg, 1.6 mmol). The solution was stirred for 15 minutes and was then added to the solution of amine hydrochloride. The solution was stirred overnight and allowed to warm to room temperature. The reaction was incomplete, so DMAP (50 mg) and the sulfonyl chloride (50 mg) were added and the reaction was stirred at room temperature for 24 hours. The reaction mixture was diluted with CH₂Cl₂ (50 mL), washed with 2N HCl (50 mL), then brine (50 mL), dried over Na₂SO₄ and concentrated to a solid (360 mg). Chromatography over silica gel using a gradient elution of EtOAc/hexane provided the title compound as a solid (293 mg) which was used as was in the next step.

### Step 7: 4-Chloro-N-[(1S,2R)-3,3,3-trifluoro-1-(hydroxymethyl)-2-phenylpropyl]benzenesulfonamide

A solution of the sulfonamide (101 mg, 0.17 mmol) from Step 6 in Et₂O/THF (2.0 mL, (1:1)) was treated with water (5 μL) and cooled to 0° C. To this was added LiBH₄ solution (100 μL, 2 M in THF). The reaction was stirred for 16 hours, quenched with 1N HCl (0.5 mL) and diluted with EtOAc (10 mL). The organic layer was separated, washed with water, dried over MgSO₄, filtered and concentrated in vacuo. Chromatography over silica gel using a gradient elution of EtOAc/hexane provided the title compound as a white solid (49 mg). A sample was recrystallized from ethyl acetate/hexane. mp 129-131° C. MS (−ESI): m/z 392.0 [M−H]⁻.

### Examples 7-8

### 5-Chloro-N-[(1S*,2S*)-2-(3,5-difluorophenyl)-3,3,3-trifluoro-1-(hydroxymethyl)propyl]thiophene-2-sulfonamide and 5-chloro-N-[(1R*,2R*)-2-(3,5-difluorophenyl)-3,3,3-trifluoro-1-(hydroxymethyl)propyl]thiophene-2-sulfonamide (7) (Method B) and 5-Chloro-N-[(1S*,2R*)-2-(3,5-difluorophenyl)-3,3,3-trifluoro-1-(hydroxymethyl)propyl]thiophene-2-sulfonamide (8) (Method A or B below)

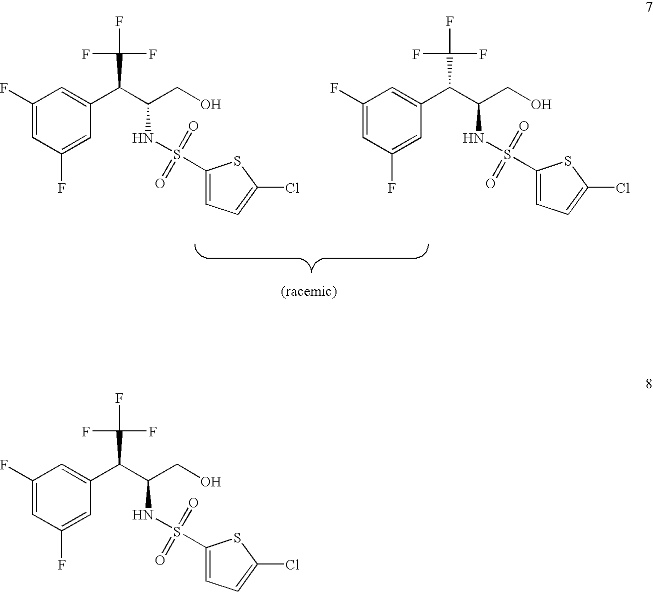

### Method A:

### Step 1: E/Z 3-(3,5-Difluoro-phenyl)-4,4,4-trifluoro-but-2-enoic acid methyl ester

1-(3,5-Difluorophenyl)-2,2,2-trifluoroethanone (1.0 g, 4.8 mmol) was added to CH₂Cl₂ (5 mL). Trimethylphosphonoacetate (0.87 g, 4.8 mmol) was added and the mixture was cooled to 0° C. Tetramethylguanidine (0.72 mL, 5.7 mmol) was added dropwise by syringe. The mixture was allowed to attain room temperature slowly and was stirred 22 hours. The reaction mixture was poured into a separatory funnel containing CH₂Cl₂ (40 mL). The organic phase was washed with distilled water (3×), brine, and then dried over Na₂SO₄. The solution was filtered and the solvents concentrated to a crude oil, which was flash chromatographed using CH₂Cl₂ as eluent. This gave the desired product as a yellow oil (1.13 g, 89%; mixture of E/Z isomers).

### Step 2: E/Z-3-(3,5-Difluorophenyl)-4,4,4-trifluorobut-2-enoic acid

E/Z 3-(3,5-Difluoro-phenyl)-4,4,4-trifluoro-but-2-enoic acid methyl ester (1.04 g, 3.90 mmol) as obtained above was dissolved in MeOH (8 mL). K₂CO₃ (2.6 g, 19 mmol) was added, followed by distilled water (8 mL). The mixture was stirred vigorously for 16 hours. The MeOH was evaporated and the aqueous residue was acidified to pH=2 using concentrated HCl. The aqueous acid phase was extracted with EtOAc (3×). The combined organic layers were dried over Na₂SO₄. Filtration and concentration of the organic phase produced an off-white crystalline solid (0.935 g, 95%) as product. The product was a mixture of E and Z isomers (60/40).

### Step 3: 4-(S)-Benzyl-3-[3-(3,5-difluoro-phenyl)-4,4,4-trifluoro-but-2-enoyl]oxazolidin-2-one

A solution of E/Z-3-(3,5-difluorophenyl)-4,4,4-trifluorobut-2-enoic acid (0.90 g, 3.6 mmol) in THF (8 mL) was cooled to −78° C. Triethylamine (0.52 mL, 3.7 mmol) and trimethylacetyl chloride (0.46 mL, 3.7 mmol) were added via syringe, in that order. The dry ice/acetone bath was replaced with an ice bath and the reaction was stirred at 0° C. for 2 hours, then the reaction was recooled to −78° C.

In a separate flask, and performed simultaneously as the above reaction, (S)-(−)-4-benzyl-2-oxazolidinone (0.63 g, 3.6 mmol) was dissolved in THF (7 mL) and cooled to −78° C., then n-butyl-lithium (1.6 M in hexanes, 2.2 mL, 3.6 mmol) was added via syringe. The reaction was stirred for 2 hours. At the end of 2 hours, this second reaction was taken up in a syringe and added to the first reaction, and stirring was allowed to continue for 1 hour at −78° C. The dry ice/acetone bath was then removed and the mixture was permitted to stir at room temperature for 16 hours.

The reaction mixture was then cooled to 0° C. and distilled water (10 mL) was added (initially with caution) followed by dilution and extraction with EtOAc (50 mL). The aqueous phase was removed and the organic phase was extracted sequentially using 1N HCl (2×20 mL), saturated NaHCO₃ solution, distilled H₂O and brine. The organic phase was dried over Na₂SO₄, filtered and concentrated to provide the desired intermediate (1.41 g, 96%). MS (+ESI): m/z 412.7 [M+H]⁺.

### Step 4: 4-(S)-Benzyl-3-[3-(3,5-difluoro-phenyl)-4,4,4-trifluoro-butyryl]oxazolidin-2-one

A standard hydrogenation bottle was charged with 4-(S)-benzyl-3-[3-(3,5-difluoro-phenyl)-4,4,4-trifluoro-but-2-enoyl]oxazolidin-2-one (1.03 g, 2.50 mmol) dissolved in EtOAc (25 mL) and 10% palladium on carbon catalyst (114 mg). The bottle was attached to a Parr hydrogenator and allowed to shake for 16 hours. The mixture was filtered through a pad of the CELITE® reagent and the solvent was evaporated to give the product as an off-white solid (1.0 g, 96%). MS (+ESI): m/z 414 [M+H]⁺.

### Step 5: (4S)-3-(2-(S)-Azido-3-(3,5-difluorophenyl)-4,4,4-trifluorobutanoyl)-4-benzyloxazolidin-2-one

The substrate, 4-(S)-benzyl-3-[3-(3,5-difluoro-phenyl)-4,4,4-trifluoro-butyryl]oxazolidin-2-one (0.10 g, 0.24 mmol), was dissolved in THF (2 mL) and cooled to −78° C. A syringe containing potassium hexamethyldisilazide 0.5 M in toluene (0.52 mL, 0.26 mmol) was cooled with dry ice and its contents added dropwise to this mixture. The solution was allowed to stir 1 hour. 2,4,6-Trisyl azide (0.082 g, 0.265 mmol) dissolved in THF (2 mL) was added quickly to the mixture via a dry ice-cooled syringe. The mixture was allowed to stir 3 minutes, then acetic acid (HOAc; 0.066 g, 1.11 mmol) was added all at once. The mixture was allowed to reach room temperature and was stirred 3 days. The reaction was diluted with EtOAc (20 mL), then washed with saturated brine (2×). The organic phase was dried over Na₂SO₄, filtered and concentrated to a crude oil which was flash chromatographed on silica gel using hexane: EtOAc, 4:1, as eluent. This afforded the product as an oil (0.053 g, 49%). MS (+ESI): m/z 454 [M]⁺.

### Step 6: (4S)-3-(2-(S)-Amino-3-(3,5-difluorophenyl)-4,4,4-trifluorobutanoyl)-4-benzyloxazolidin-2-one hydrochloride salt

A standard hydrogenation bottle was charged with 10% palladium on carbon catalyst (0.006 g), and (4S)-3-(2-(S)-Azido-3-(3,5-difluorophenyl)-4,4,4-trifluorobutanoyl)-4-benzyloxazolidin-2-one (0.038 g, 0.083 mmol) dissolved in MeOH (3 mL) was added. Propionyl chloride (0.015 g, 0.167 mmol) dissolved in MeOH (2 mL) was then added. The bottle was attached to a Parr hydrogenator and allowed to shake for 16 hours. The mixture was filtered through a pad of the CELITE® reagent and the solvent was evaporated to give the product as a pale yellow solid that was used in the next procedure without further purification.

### Step 7: N—(S)-(1-((S)-4-Benzyl-2-oxooxazolidin-3-yl)-3-(3,5-difluorophenyl)-4,4,4-trifluoro-1-oxobutan-2-yl)-5-chloro-thiophene-2-sulfonamide

To a mixture of 5-chloro-thiophene-2-sulfonyl chloride (0.028 g, 0.130 mmol) and dimethylaminopyridine (0.018 g, 0.143 mmol) in CH₂Cl₂ (0.5 mL) was added (4S)-3-(2-(S)-amino-3-(3,5-difluorophenyl)-4,4,4-trifluorobutanoyl)-4-benzyloxazolidin-2-one hydrochloride salt (0.028 g, 0.065 mmol) as a solution dissolved in CH₂Cl₂ (0.5 mL). The mixture was allowed to stir for 24 hours. The reaction was diluted with CH₂Cl₂ (10 mL) and washed with distilled water (2×). The organic phase was dried (MgSO₄), filtered and evaporated to a crude oil that was flash chromatographed using hexane:ethyl acetate (EtOAc), 4:1, as eluent. This gives the desired intermediate as an oil (0.020 g, 51%). MS (+ESI): m/z 609 [M+H]⁺.

### Step 8: 5-Chloro-N-[(1S*, 2R*)-2(3,5-difluorophenyl)-3,3,3-trifluoro-1-(hydroxymethyl)propyl]-thiophene-2-sulfonamide (8)

To the substrate, N—(S)-(1-((S)-4-benzyl-2-oxooxazolidin-3-yl)-3-(3,5-difluorophenyl)-4,4,4-trifluoro-1-oxobutan-2-yl)-5-chloro-thiophene-2-sulfonamide (0.0195 g, 0.032 mmol) dissolved in anhydrous THF (0.3 mL) was added LiBH₄ (2 M in THF, 0.032 mL, 0.064 mmol) by syringe under a nitrogen atmosphere. The mixture was allowed to stir 16 hours. The reaction was quenched by cautious addition of 2N HCl solution until gas evolution ceased (3-4 drops). The mixture was diluted with EtOAc (10 mL) and distilled water (2 mL). The organic phase was separated and the aqueous phase was washed again with EtOAc. The combined organic layers were dried over MgSO₄, filtered and concentrated to produce a crude oil that was separated by flash chromatography on silica gel using a gradient elution of EtOAc/hexanes followed by chiral preparative HPLC (using the method of Example 5) to afford the desired product as an oil (0.002 g) in 14% yield. MS (−ESI): m/z 434 [M−H]⁻.

### Method B:

### Step 1: 3-(3,5-Difluoro-phenyl)-4,4,4-trifluoro-2-formylamino-but-2-enoic acid ethyl ester

(See Enders, D. et al.; Synthesis (2005) pages 306-310.)

Potassium t-butoxide (95%; 0.811 g, 6.86 mmol) was suspended in dry THF (5 mL). The suspension was cooled to −78° C. A solution of ethyl isocyanoacetate (95% pure; 0.79 mL, 6.86 mmol) dissolved in THF (1.5 mL) was then added dropwise. After the addition, the mixture was stirred at −78° C. for 30 minutes. A solution of 1-(3,5-difluoro-phenyl)-2,2,2-trifluoro-ethanone (1.441 g, 6.86 mmol) dissolved in THF (2 mL) was then added dropwise to the mixture and the reaction was allowed to stir for 1 hour at −78° C. The cooling bath was removed, and the mixture allowed to warm at room temperature for 2 hours. 1N HCl (6.9 mL) was added and the mixture was stirred for 30 minutes at room temperature. The THF layer was decanted and retained and the aqueous phase was extracted twice with CH₂Cl₂. The combined organic phase was dried (Na₂SO₄) and evaporated. The crude material was chromatographed on silica gel using EtOAc/CH₂Cl₂ in a gradient elution (0-3% EtOAc) to afford the title olefin as a single (Z) isomer (1.749 g, 5.41 mmol; 79%).

### Step 2: N-[2-(3,5-Difluoro-phenyl)-3,3,3-trifluoro-1-hydroxymethyl-propyl]-formamide

3-(3,5-Difluoro-phenyl)-4,4,4-trifluoro-2-formylamino-but-2-enoic acid ethyl ester (1.740 g, 5.39 mmol) was dissolved in MeOH (11 mL). Sodium borohydride (0.611 g, 16.2 mmol) was added in portions and then the mixture was allowed to stir for 2 hours at room temperature. The solvent was evaporated, and the residue was taken up in dry THF (7 mL). Lithium borohydride (2 M in THF, 6.73 mL, 13.5 mmol) was then added slowly via syringe, and the mixture was allowed to stir for 2.5 hours at room temperature. The reaction was quenched carefully with 2N HCl until bubbling subsided. A small amount of water and diethyl ether was added to help keep the suspension fluid. The mixture was then extracted with CH₂Cl₂ (2×) and EtOAc (1×). (Note: On subsequent runs switching the order of the ethyl acetate and CH₂Cl₂ extractions avoided some emulsion problems.) The combined organic extracts were dried (MgSO₄) and evaporated. The crude material was chromatographed on silica gel using MeOH/CH₂Cl₂ in a gradient elution (1-5% MeOH) to afford the title compound as a mixture of diastereomers (about 1.75:1 ratio; 0.834 g, 2.95 mmol; 55%).

### Step 3: 5-Chloro-N-[(1S*, 2R*)-2-(3,5-difluorophenyl)-3,3,3-trifluoro-1-(hydroxymethyl)propyl]thiophene-2-sulfonamide

The diastereomeric mixture of N-[2-(3,5-difluoro-phenyl)-3,3,3-trifluoro-1-hydroxymethyl-propyl]-formamide obtained above (0.812 g, 2.87 mmol) was dissolved in 3N HCl/MeOH (20 mL), and the solution was allowed to stir for 4.5 hours at room temperature. The pH of the solution was adjusted to about 10 with about 25% NaOH and the mixture was extracted thrice with CH₂Cl₂ (the water phase was “salted out” prior to extraction the second and third time). The combined organic phase was dried (MgSO₄) and evaporated to afford the crude, expected amine (705 mg).

5-Chlorothiophene-2-sulfonyl chloride (388 μL, 3.01 mmol) was dissolved in CH₂Cl₂ (2.1 mL) and 4-dimethylaminopyridine (0.372 g, 3.16 mmol) was added. The mixture was allowed to stir for 5 minutes and the above amine (705 mg) dissolved in CH₂Cl₂ (2 mL) was added. The mixture was allowed to stir overnight at room temperature. The mixture was diluted with CH₂Cl₂ and washed with 2N HCl (2×), water, and then brine. The organic phase was dried (MgSO₄) and evaporated to afford the crude mixture of products. The material was chromatographed on silica gel using a gradient elution of EtOAc/CH₂Cl₂ (5-20% EtOAc) to afford a racemic mixture of two diastereomers (about 1.75:1 ratio; 794 mg, 1.83 mmol; 64%). The material was further purified by chiral preparative HPLC to afford 5-chloro-N-[(1S*,2S*)-2-(3,5-difluorophenyl)-3,3,3-trifluoro-1-(hydroxymethyl)propyl]thiophene-2-sulfonamide and 5-chloro-N-[(1R* ,2R*)-2-(3,5-difluorophenyl)-3,3,3-trifluoro-1-(hydroxymethyl)propyl]thiophene-2-sulfonamide (7) (0.359 g, 0.825 mmol; 30%) along with 5-chloro-N-[(1S*,2R*)-2-(3,5-difluorophenyl)-3,3,3-trifluoro-1-(hydroxymethyl)propyl]thiophene-2-sulfonamide (8) (0.119 g, 0.274 mmol; 10%). (7) MS (−ESI): m/z 434 [M−H]⁻. (8) MS (−ESI): m/z 434 [M−H]⁻. CHIRALPREP® LC Conditions: VARIAN® Prep LC; the CHIRALCEL® AD column (5×50 cm); Mobile phase 15% ethanol in hexane; Flow rate 100 mL/min. Chiral Analytical LC analysis: the Chiralcel® AD-H column.

*Absolute stereochemistry of 8 was confirmed by single-crystal X-ray analysis.

### Method C:

Compound 8 (1.32 g; mixture of isomers) was prepared according to Method B and then subsequently purified using the following conditions: Varian™ Prep LC; Chiralcel® AD column (2×25 cm); Mobile phase 10% ethanol in hexane; Flow rate 22 mL/min. Chiral Analytical LC analysis: a Chiralcel® AD-H column. Compound 8 (0.360 g) so obtained contained about five percent of total impurities. A sample of the above material (0.230 g) was further purified using the following conditions: Varian™ Prep LC; Primesphere® C18 column (5×25 cm); Mobile phase 57% acetonitrile in 10 mM ammonium acetate; Flow rate 85 mL/min. This afforded 0.190 g of compound 8 that was 99.9% chemically and chirally pure.

### Examples 9-10

### 5-Chloro-N-[(1S*,2R*)-3,3,3-trifluoro-2-(4-fluorophenyl)-1-(hydroxymethyl)propyl]thiophene-2-sulfonamide (9) and 5-Chloro-N-[(1S*,2S*)-3,3,3-trifluoro-2-(4-fluorophenyl)-1-(hydroxymethyl)propyl]thiophene-2-sulfonamide (10)

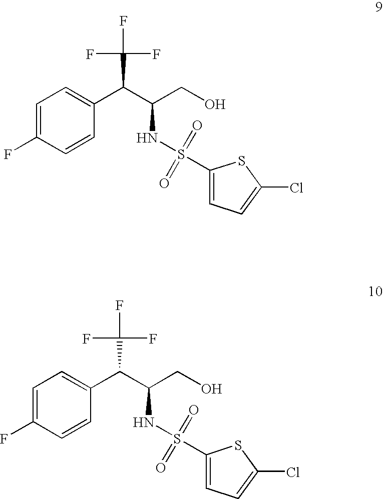

### Step 1: 4,4,4-Trifluoro-3-(4-fluoro-phenyl)-but-2-enoic acid tert-butyl ester

A solution of 4′-fluoro-2,2,2-trifluoroacetophenone (10 g, 52 mmol) and (dimethoxy-phosphanyloxy)-acetic acid tert-butyl ester (11.6 g, 52.4 mmol) in CH₂Cl₂ (50 mL) was treated with tetramethylguanidine (6.3 g, 52.4 mmol) at room temperature for 48 hours. The solution was extracted with 1N HCl, and then washed with brine. The organic layer was dried over MgSO₄, filtered and concentrated in vacuo to provide an oil (14.6 g; 96%) which was a 1:1 mixture of olefin isomers. ¹H NMR (CDCl₃): δ 6.51 (d, 0.5 H, J=1.4 Hz), 6.22 (s, 0.5 H), 1.5 (s, 4.5 H), 1.23 (s, 4.5 H).

### Step 2: 4,4,4-Trifluoro-3-(4-fluoro-phenyl)-but-2-enoic acid

To a well stirred 0° C. solution of sulfuric acid (2.45 g, 25 mmol) in CH₂Cl₂ (90 mL) was added a solution of 4,4,4-trifluoro-3-(4-fluoro-phenyl)-but-2-enoic acid tert-butyl ester (14.6 g, 50.3 mmol). The solution was allowed to warm to room temperature over 4 hours and stirring continued for 18 hours. The solution was carefully treated with NaOH solution (40 mL, 2.5 N). The layers were separated and the organic layer was washed with additional NaOH (40 mL, 2.5 N). The aqueous layers were pooled and the pH was adjusted to 1 with concentrated HCl. The aqueous mixture was extracted with diethyl ether (2×50 mL). The organic layers were pooled and washed with 10% Na₂SO₄ (1×25 mL), dried over Na₂SO₄, filtered, and concentrated in vacuo. This provided 9.13 g of the product as a mixture of olefins. ¹H NMR (CDCl₃): δ 10.68 (s, 1 H), 6.58 (s, 0.5 H), 6.29 (s, 0.5 H).

### Step 3: (S)-4-Benzyl-3-[4,4,4-trifluoro-3-(4-fluoro-phenyl)-but-2-enoyl]-oxazolidin-2-one

A solution of 4,4,4-trifluoro-3-(4-fluoro-phenyl)-but-2-enoic acid (8.47 g, 36.2 mmol) in THF (200 mL) was cooled to −78° C. To this was added triethylamine (5.8 mL, 40.9 mmol) followed by pivaloyl chloride (4.60 mL, 37.1 mmol). This solution was stirred at −78° C. for 30 minutes, then allowed to warm to room temperature for 1 hour. The solution was then recooled to −78° C.

A solution of (S)-4-benzyl-oxazolidinone (7.04 g, 39.7 mmol) and triphenylmethane (25 mg) in THF (200 mL) was cooled to −78° C. To this was added n-BuLi (1.64 M in hexane) until the solution turned red (approximately 25 mL). This solution was stirred for 30 minutes at −78° C., and then the anion solution was added to the cold mixed anhydride. The mixture was stirred overnight and allowed to warm to room temperature. The solution was recooled, quenched with saturated aqueous Na₂SO₄ and warmed to room temperature. The organic solvent was removed in vacuo and the material was partitioned between EtOAc and water. The layers were separated and the aqueous layer was extracted twice with EtOAc. The pooled organic layers were washed with 1N HCl, saturated sodium bicarbonate solution then saturated brine. The organic phase was dried over MgSO₄, filtered and concentrated in vacuo to yield 15.6 g of an orange oil. The material was chromatographed on silica gel using a gradient elution of EtOAc/hexane to yield the title compound as a white solid. MS (APPI): m/z 394 [M+H]⁺.

### Step 4: (S)-4-Benzyl-3-[4,4,4-trifluoro-3-(4-fluoro-phenyl)-butyryl]-oxazolidin-2-one

A solution of (S)-4-benzyl-3-[4,4,4-trifluoro-3-(4-fluoro-phenyl)-but-2-enoyl]-oxazolidin-2-one (720 mg, 1.8 mmol) in EtOH (50 mL) was hydrogenated over 10% Pd/C (72 mg) at 1 atmosphere for 16 hours. The sample was filtered through the CELITE® reagent and the CELITE® reagent was rinsed with EtOH (2×25 mL). The solvent was removed in vacuo to yield 670 mg of the title compound as an oil which solidified on standing. This mixture was used as is in the next step. ¹H NMR (CDCl₃): δ 3.87 (dd, 0.5 H), 3.67 (dd, 0.5 H), 3.55 (dd, 0.5 H), 3.44 (dd, 0.5 H).

### Step 5: 3-[(S)-2-Azido-4,4,4-trifluoro-3-(4-fluoro-phenyl)-butyryl]-(S)-4-benzyl-oxazolidin-2-one

A solution of (S)-4-benzyl-3-[4,4,4-trifluoro-3-(4-fluoro-phenyl)-butyryl]-oxazolidin-2-one (670 mg, 1.69 mmol) in THF (25 mL) was cooled to −78° C. under N₂. To this was added a solution of potassium bis(trimethylsilyl)amide (3.5 mL, 0.5 M solution in toluene). This mixture was stirred at −78° C. for 45 minutes. A solution of trisyl azide (410 mg, 2.0 mmol) in THF (3 mL) was prepared and cooled to −78° C. This solution was added to the anion solution via a cannula. After 3 minutes, the reaction was quenched with HOAc (0.57 mL, 10 mmol). The reaction was warmed to room temperature over 3 hours. The solvents were removed in vacuo and the residue was partitioned between EtOAc and brine. The aqueous layer was extracted with EtOAc and the organic layers were pooled, dried over MgSO₄, filtered and concentrated in vacuo to yield 0.95 g of an oil. Chromatography over silica gel using a gradient elution of EtOAc/hexane provided 480 mg of the title compound as an oil.

### Step 6: (S)-2-Azido-4,4,4-trifluoro-3-(4-fluoro-phenyl)-butan-1-ol

A solution of 3-[(S)-2-azido-4,4,4-trifluoro-3-(4-fluoro-phenyl)-butyryl]-(S)-4-benzyl-oxazolidin-2-one (480 mg, 1.1 mmol) in THF (50 mL) was treated with water (0.2 mL) then cooled to 0° C. To this solution was added LiBH₄ (1.5 mL, 2 M in THF). The reaction was stirred overnight and allowed to warm to room temperature overnight. The reaction was quenched with 1N HCl (2 mL). The solvents were removed and the residue was partitioned between EtOAc and water. The organic layer was washed with saturated sodium bicarbonate solution followed by brine. The organic layer was dried over MgSO₄, filtered, and concentrated in vacuo to an oil. The oil was chromatographed over silica gel using a gradient elution of EtOAc/hexane to yield 220 mg of the title compound as an oil. This material was used as is in the next step.

### Step 7: (S)-2-Amino-4,4,4-trifluoro-3-(4-fluoro-phenyl)-butan-1-ol hydrochloride

A solution of (S)-2-azido-4,4,4-trifluoro-3-(4-fluoro-phenyl)-butan-1-ol (200 mg, 0.8 mmol) was dissolved in MeOH (10 mL) and ethanolic HCl (1 mL, 1.25 M). This mixture was hydrogenated at 1 atmosphere over 10% Pd/C (25 mg) for 24 hours. The solution was filtered through the CELITE® reagent and the solvent was removed in vacuo to yield the salt as a glass. This material was used without further purification.

### Step 8: 5-Chloro-N-[(1S*,2R*)-3,3,3-trifluoro-2-(4-fluorophenyl)-1-(hydroxymethyl)propyl]thiophene-2-sulfonamide (9) and 5-Chloro-N-[(1S*,2S*)-3,3,3-trifluoro-2-(4-fluorophenyl)-1-(hydroxymethyl)propyl]thiophene-2-sulfonamide (10)

A solution of (S)-2-amino-4,4,4-trifluoro-3-(4-fluoro-phenyl)-butan-1-ol hydrochloride (200 mg, 0.73 mmol) in CH₂Cl₂ (15 mL) was treated with 1-methyl-morpholine (0.4 mL, 3.6 mmol) and then chlorotrimethylsilane (0.12 mL, 1.5 mmol). The reaction was stirred for 15 minutes, cooled to 0° C. and was treated with 5-chloro-thiphene-2-sulfonyl chloride (0.180 g, 0.83 mmol). The reaction was stirred and allowed to warm to room temperature overnight. The reaction was diluted with EtOAc (25 mL) and was treated with 2N HCl (10 mL) for 10 minutes. The aqueous layer was extracted with EtOAc (25 mL) and the organic layers were pooled, dried over MgSO₄, filtered, concentrated in vacuo and chromatographed on silica gel using a gradient elution of EtOAc/hexane to yield the product (50 mg) as an oil. Subsequent chromatography on the CHIRALCEL® AD-H column yielded 24 mg of the 1S,2R isomer (9) as an amorphous solid, and 18 mg of the 1S,2S isomer (10) was obtained as well. (9) MS (−ESI): m/z 415.9 [M−H]⁻. HRMS: calcd for C₁₄H₁₂ClF₄NO₃S₂—H⁺, 415.9805; found (ESI, [M−H]⁻), 415.9816. (10) MS (−ESI): m/z 415.9 [M−H]⁻. HRMS: calcd for C₁₄H₁₂ClF₄NO₃S₂—H⁺, 415.9805; found (ESI, [M−H]⁻), 415.9804. Absolute stereochemistry of 9 was assigned by analogy to the compound of Example 4. The X-ray data of Example 4 and NMR data were used to assign active stereoisomers which were consistent with the biological data. The absolute stereochemistry of 10 was assigned based upon the compound being the diastereomer of 9.

### Examples 11-12

### 5-Chloro-N-[(1S*,2S*)-3,3,3-trifluoro-2-(3-fluorophenyl)-1-(hydroxymethyl)propyl]thiophene-2-sulfonamide (11) and 5-Chloro-N-[(1S*,2R*)-3,3,3-trifluoro-2-(3-fluorophenyl)-1-(hydroxymethyl)propyl]thiophene-2-sulfonamide (12)

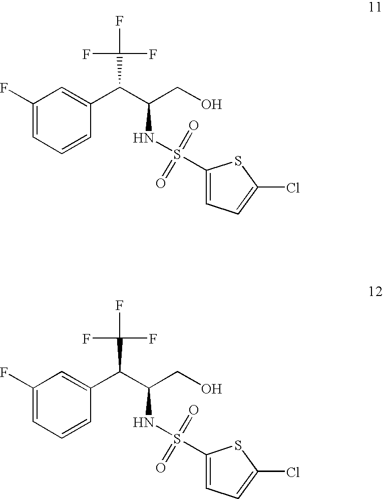

These compounds were prepared from commercially available 2,2,2-trifluoro-1-(3-fluoro-phenyl)-ethanone in exactly the same manner as the compound of Example 8 using Method B. Yields: Step 1, 66%; Step 2, 47%; Step 3, 1^{st} chromatography (silica gel), 53% of mixture of 4 isomers; Step 3, second chromatography (chiral prep LC), 6% of 11 and 7% of 12. (11) MS (−ESI): m/z 416 [M−H]⁻. HRMS: calcd for C₁₄H₁₂ClF₄NO₃S₂—H⁺, 415.9805; found (ESI, [M−H]⁻), 415.9820. (12) MS (−ESI): m/z 416 [M−H]⁻. HRMS: calcd for C₁₄H₁₂ClF₄NO₃S₂—H⁺, 415.9805; found (ESI, [M−H]⁻), 415.9822. The absolute stereochemistry of 11 and 12 was assigned by analogy to the compound of Example 4. Specifically, the X-ray data of Example 4 and NMR data were used to assign active stereoisomers which were consistent with the biological data. The absolute stereochemistry of 11 and 12 were assigned arbitrarily, but is supported by trends in biological activity.

### Examples 13-14

### 5-Chloro-N-[(1S*,2R*)-2-(4-chlorophenyl)-3,3,3-trifluoro-1-(hydroxymethyl)propyl]thiophene-2-sulfonamide (13) and 5-Chloro-N-[(1S*,2S*)-2-(4-chlorophenyl)-3,3,3-trifluoro-1-(hydroxymethyl)propyl]thiophene-2-sulfonamide (14)

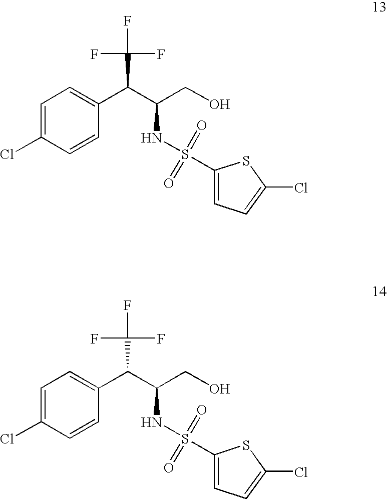

This compound was prepared from commercially available 1-(4-chloro-phenyl)-2,2,2-trifluoro-ethanone in exactly the same manner as the compound of Example 8 using Method B. However, upon evaporation of the organic phase to yield the crude product mixture from Step 3, a precipitate formed. The precipitate was collected and the mother liquor was chromatographed on silica gel according to Example 8, Method B, Step 3. The material obtained from the chromatographic fractions containing all isomers of the desired product were combined with the precipitate from the evaporation step and then subjected to chiral preparative HPLC (using the method of Example 8, Method B) to afford the title compound. Yields: Step 1, 69%; Step 2, 46%; Step 3, 1^{st} chromatography (silica gel) and precipitate, 73%; Step 3, second chromatography (chiral prep HPLC), 7% of 13. A third chiral prep LC separation was needed to obtain 7% of 14. MS (−ESI): m/z 432 [M−H]⁻. Absolute stereochemistry of 13 was assigned by analogy to the compound of Example 4. The X-ray data of Example 4 and NMR data were used to assign active stereoisomers which were consistent with the biological data. The absolute stereochemistry of 14 was assigned arbitrarily, but supported by trends in biological activity.

### Example 15

### Analysis of 5-Chloro-N-[(1S,2S)-2-(3,5-difluorophenyl)-3,3,3-trifluoro-1-(hydroxymethyl)propyl]thiophene-2-sulfonamide

A sample of 5-Chloro-N-[(1S,2S)-2-(3,5-difluorophenyl)-3,3,3-trifluoro-1-(hydroxymethyl)propyl]thiophene-2-sulfonamide prepared as described in Example 8, Method C was analyzed using powder X-ray diffraction.

X-Ray diffraction data was acquired using a D8 ADVANCE® X-ray powder diffractometer (Bruker) having the following parameters and the X-ray diffraction pattern was obtained. See, FIG. 1.

| | |
|---|---|
| voltage: | 40 kV; |
| current: | 40.0 mA; |
| scan range (2θ): | 5 to 35°; |
| scan step size: | 0.01°; |
| total scan time: | 33 minutes; |
| detector: | VANTEC ™ detector; and |
| antiscattering slit: | 1 mm. |

### Example 16

The activity of compounds in lowering the generation of Aβ40 and Aβ42, the most abundant forms of beta amyloid, from APP (amyloid precursor protein) was measured in an electrochemiluminescent (ECL) assay of conditioned media from chinese hamster ovary (CHO) cells stably expressing the human amyloid precursor protein (APP) reporter construct, hAPP-REPNL₇₅₁ (Sughir, R. et al.; J. Biolog. Chem (1992) 267: 25602-25608). Aβ peptides were secreted at high levels into cell culture media by cells carrying the human APP transgene; compounds were tested for their capacity to modulate this production. The Aβ peptide in the conditioned medium was quantitated by a sandwich immunoassay with the MSD ECL detection system. Cell metabolism was measured using a 3-(4,5-dimethylthiazol-2-yl)-5-(3-carboxymethoxypheny)-2-(4-sulfophenyl)-2H-tetrazolium, inner salt (MTS) salt kit (which measures the mitochondrial activity of cells by the bioreduction of Owen's reagent). This assay consists of the following protocol:

### A. Aβ-Lowering Assay:

1. hAPP-REPNL₇₅₁CHO cells were seeded into 96 well plates and incubated until about 60-70% confluence.

2. Medium was removed, cells were washed, and fresh serum-free medium (Ultraculture) was put on cells.

3. Compounds were diluted and then added to the cell medium.

4. Cells were incubated, with compounds, for indicated times.

5. Streptavidin coated Meso Scale Discover (MSD) plates (MSD standard MULTI-ARRAY® 96 plate, cat.# P11SA-1) were washed 333 with TTBS (Tris-buffered saline, the TWEEN® 20 reagent).

6. 20 μL conditioned medium were removed from the cells and added to the TTBS prewashed MSD plates.

7. Standard curve dilutions of synthetic Aβ40 and Aβ42 were prepared and added to the MSD plates.

8. A reagent mix was prepared in 1% MSD Blocker A (appropriate concentration of biotinylated 6E10 antibody, detection antibodies to Aβ40, Aβ42, and MSD ruthinylated tag antibody).

9. 20 μL reagent mix was distributed to sample plate.

10. Plates were incubated with shaking overnight at 4° C.

11. Plates were washed 3× with TTBS.

12. 150 μL read buffer was added per well (MSD® Read Buffer T, cat#R92TC-2, made 1× with distilled water).

13. Plates were read in a MSD® plate reader within 2 hours.

### B. MTS Assay:

1. Cells were seeded into 96 well plates and incubated until about 60-70% confluence.

2. Medium was removed, cells were washed, and fresh serum-free medium (Ultraculture) was put on cells.

3. Compounds were diluted and then added to the cell medium.

4 Cells were incubated, with compounds, for indicated times.

5. Using a robot, conditioned medium was removed from the cells, and transferred to a TTBS prewashed MSD® plate (see #6 above).

6. Cells were washed 2× in phosphate buffered saline (PBS) and an MTS solution was plated onto the cells. After 1 hour, they were read on a plate reader at 560 nm to determine metabolic activity.

### C. Analysis of Results:

Assays were accepted or rejected based upon specific performance criteria, including regression coefficient of standard curve, adequate signal to noise ratio, sample signals lying within the range of the standard curve, etc.: the specific parameters were established for each tissue type prior to performing an assay, and were included in the full analytical procedure.

Plate data from both assays (MTS and MSD® ELISA) were transferred into a Microsoft EXCEL® spreadsheet to determine the toxicity, and inhibition caused by the compounds. Standard curves of Aβ were generated using LSW™ toolbar Hill slope model 42, with 1/y weighting (general sigmoidal curve with Hill slope, a to d; y=(a−d)/(1+(x/c)̂b)+d). Inhibition data were expressed as a percent of the average of the Aβ values in the vehicle treated wells (all values are background subtracted), using LSW™ toolbar Hill Slope model 68 (ligand-receptor binding/sigmoidal, with Hill slope and Bmax to 0; y=Bmax*(1−(x̂n/(K̂n+x̂n))), after raw values were translated/transformed into absolute Aβ values utilizing the standard curve generated as described above (e.g. back calculate absolute Aβ values from the raw values utilizing standard curve). Onboard controls to verify assay performance were checked to assure that amyloid is within linear detection range of the assay, that cells were expressing correctly, and that the MSD itself was performing according to quality control (QC) standards.

### D. Response Determination:

% Inhibition>50% may be considered a positive response or an interesting result in this assay; EC₅₀ determination were determined.

### E. Beta Amyloid Assay Reference Compounds:

5-chloro-N-[(1S,2R)-2-ethyl-4,4,4-trifluoro-1-(hydroxymethyl)butyl]thiophene-2-sulfonamide (EC₅₀Aβ40=6 nM, EC₅₀Aβ42=5 nM; International Patent Publication No. WO 2004/092155, data not shown. These data were generated using Example 15).

(2S)-2-hydroxy-3-methyl-N-((1S)-1-methyl-2-{[(1S)-3-methyl-2-oxo-2,3,4,5-tetrahydro-1H-3-benzazepin-1-yl]amino}-2-oxoethyl)butanamide (EC₅₀Aβ40=109 nM, EC₅₀Aβ42=79 nM, International Patent Publication No. WO 2002/47671, data not shown). These data were generated using Example 15.

A concern with compounds that lower beta amyloid synthesis by targeting the γ-secretase enzyme is that this enzyme also is involved in the cleavage of other substrates besides APP especially the Notch substrate. Inhibition of Notch cleavage has been found to cause multiple side effects, including failure of T cell differentiation and lesions of the gastrointestinal tract. Thus, a selectivity of APP cleavage over Notch cleavage is desired in order to avoid G.I. side effects. The activity of compounds in cleaving Notch can be measured in a whole cell functional Notch assay. This is an assay to determine the effects of compounds on the S3 (γ-secretase like) processing of Notch. The assay permits measurement of the inhibition of S3 cleavage activity as revealed by reduced transactivation of a reporter gene: specifically, a constitutively active form of Notch (having the extracellular domain deletion) when cleaved by γ-secretase releases the Notch intracellular domain (NICD) which transactivates the soluble alkaline phosphatase (SEAP) gene driven by the HES promoter. SEAP transactivation is then detected by luminescent assay. The assay consists of the following protocol:

### Materials and Methods:

(i) Materials: The P8H6 inducible stable cell line was derived from T-REX™ CHO-K1, stably expressing Tet repressor, Notch (pZX1) and HES1-SEAP (pZX2) reporter constructs. A Wallac 1450 Victor™ Luminescence Counter was used to measure luminescent signal. All other materials and reagents were of highest quality available and were all from commercial sources.

(ii) Construct Description:
pZX1: Mouse Notch1 with deleted extracellular domain cloned at Hind III/Xho I sites of pcDNA5/TO vector: Ampicillin Resistance for bacteria, hygromycin resistance for cells.
pZX2: Mouse HES1 promoter together with reporter gene SEAP cloned at Not I/Apa I sites of pcDNA3.1 with deletion of CMV promoter.

(iii) Procedure:
1. P8H6 cells were grown in Ham's F12 complete growth media containing 10% FBS (reduced tetracycline), 2 mM of L-glutamine, 1 mg/mL of hygromycin, 1 mg/mL of the GENETICIN® antibiotic, 10 μg/mL of Blasticidin and 1% Penicillin-Streptomycin were supplemented with doxycline with final induction concentration of 0.2 μg/mL. The cells were seeded in 96-well tissue culture plate at 8000/well.
2. Test compounds in 0.2% DMSO/25 mM HEPES buffer were diluted at final working concentration of 20 μM, 6.7 μM, 2.2 μM, 741 nM, 247 nM, 82 nM, 27 nM, 9 nM and 3 nM. Diluted compounds then were added to P8H6 cells in 96-well tissue culture plates. For vehicle samples, the same volume of 0.2% DMSO/25 mM HEPES buffer was added.
3. Cells were incubated at 37° C., 5% CO₂ for 48 hours.
4. SEAP levels in the conditioned media were assessed using the Clontech GREATESCAPE™ SEAP Chemiluminescence detection kit according to the manufacturer's instruction. Briefly, 15 μL of conditioned media were mixed with 45 μL of the dilution buffer and incubated at 65° C. for 45 minutes. After cooling, assay buffer was added and the sample was incubated with the CSPD® substrate.
5. Luminescence was measured in a Wallac 1450 VICTOR™ luminescence counter.

### Analysis Of Results:

EC₅₀ was the compound concentration that was estimated to provide a 50% reduction of maximum response in Notch induced SEAP levels. An EC₅₀ from a given assay may only be used for averaging if it has met the following criteria:
1. There is a lower dose in the assay which gives a % Notch Activity>60% (bracketing lower dose).
2. There is a higher dose in the assay which gives a % Notch Activity<30% (bracketing higher dose), and the maximum inhibition is reached (e.g., a given compound might achieve 80% inhibition (% Notch activity is about 20%) over several doses, and therefore 20% would be considered the maximum inhibition.

Data were analyzed in Microsoft EXCEL® format. EC₅₀ was calculated by LSW using sigmoidal inhibition from B0 to nsb model (model 59). Each assay plate contained a dose response curve for the reference compound 5-chloro-N-[(1S,2R)-2-ethyl-4,4,4-trifluoro-1-(hydroxymethyl)butyl]thiophene-2-sulfonamide. This compound is discussed in International Patent Publication No. WO 2004/092155. The following data is not shown in that publication. The EC₅₀ for this reference compound must fall within the range of 150-350 nM for the assay plate to be accepted.

F. Notch Assay reference Compounds:

5-chloro-N-[( 1S,2R)-2-ethyl-4,4,4-trifluoro-1-(hydroxymethyl)butyl]thiophene-2-sulfonamide (EC₅₀=225 nM).

(2S)-2-hydroxy-3-methyl-N-((1S)-1-methyl-2-{[(1S)-3-methyl-2-oxo-2,3,4,5-tetrahydro-1H-3-benzazepin-1-yl]amino}-2-oxoethyl)butanamide (EC₅₀=68 nM). This compound is discussed in International Patent Publication No. WO 2002/47671, but this data is not shown.

The beta amyloid inhibitory activity of compounds of formula (I) was determined using the MSD ECL assay. Inhibition of Notch processing was measured using the stable transfection reporter assay See, Table 1 below.

TABLE 1
Biological Data

| Example | Aβ40* | Aβ42* | Notch^{θ} | Compound |
|---|---|---|---|---|
| 1 | 710 | 760 | >20,000 | 5-chloro-N-[3,3,3-trifluoro-1-(hydroxymethyl)-2- |
| | | | | phenylpropyl]thiophene-2-sulfonamide |
| 2 | >15,000 | >15,000 | | 5-chloro-N-[(1R,2R)-3,3,3-trifluoro-1- |
| | | | | (hydroxymethyl)-2-phenylpropyl]thiophene-2- |
| | | | | sulfonamide |
| 3 | >15,000 | >15,000 | | 5-chloro-N-[(1R,2S)-3,3,3-trifluoro-1- |
| | | | | (hydroxymethyl)-2-phenylpropyl]thiophene-2- |
| | | | | sulfonamide |
| 4 | 238 | 197 | 3753 | 5-chloro-N-[(1S,2R)-3,3,3-trifluoro-1- |
| | | | | (hydroxymethyl)-2-phenylpropyl]thiophene-2- |
| | | | | sulfonamide |
| 5 | 7517 | 7205 | >20,000 | 5-chloro-N-[(1S,2S)-3,3,3-trifluoro-1-(hydroxymethyl)- |
| | | | | 2-phenylpropyl]thiophene-2-sulfonamide |
| 6 | 1326 | 1899 | >20,000 | 4-chloro-N-[(1S,2R)-3,3,3-trifluoro-1- |
| | | | | (hydroxymethyl)-2-phenylpropyl]benzenesulfonamide |
| 7 | 7295 | 8754 | >20,000 | 5-chloro-N-[(1S,2S)-2-(3,5-difluorophenyl)-3,3,3- |
| | | | | trifluoro-1-(hydroxymethyl)propyl]thiophene-2- |
| | | | | sulfonamide and 5-chloro-N-[(1R,2R)-2-(3,5- |
| | | | | difluorophenyl)-3,3,3-trifluoro-1- |
| | | | | (hydroxymethyl)propyl]thiophene-2-sulfonamide |
| 8 | 83 | 80 | 2778 | 5-chloro-N-[(1S,2R)-2-(3,5-difluorophenyl)-3,3,3- |
| | | | | trifluoro-1-(hydroxymethyl)propyl]thiophene-2- |
| | | | | sulfonamide |
| 9 | 187 | 178 | >20,000 | 5-chloro-N-[(1S,2R)-3,3,3-trifluoro-2-(4-fluorophenyl)- |
| | | | | 1-(hydroxymethyl)propyl]thiophene-2-sulfonamide |
| 10 | 1876 | 1572 | >20,000 | 5-chloro-N-[(1S,2S)-3,3,3-trifluoro-2-(4-fluorophenyl)- |
| | | | | 1-(hydroxymethyl)propyl]thiophene-2-sulfonamide |
| 11 | 4062 | 3382 | >20,000 | 5-chloro-N-[(1S,2S)-3,3,3-trifluoro-2-(3-fluorophenyl)- |
| | | | | 1-(hydroxymethyl)propyl]thiophene-2-sulfonamide |
| 12 | 98 | 98 | 1739 | 5-chloro-N-[(1S,2R)-3,3,3-trifluoro-2-(3-fluorophenyl)- |
| | | | | 1-(hydroxymethyl)propyl]thiophene-2-sulfonamide |
| 13 | 316 | 396 | 7602 | 5-chloro-N-[(1S,2R)-2-(4-chlorophenyl)-3,3,3- |
| | | | | trifluoro-1-(hydroxymethyl)propyl]thiophene-2- |
| | | | | sulfonamide |
| 14 | 2364 | 2284 | >20,000 | 5-chloro-N-[(1S,2S)-2-(4-chlorophenyl)-3,3,3-trifluoro- |
| | | | | 1-(hydroxymethyl)propyl]thiophene-2-sulfonamide |

EC₅₀ for inhibition of Aβ40/Aβ42 in nM as determined by MSD electrochemiluminescent assay.
^{θ}EC₅₀ for inhibition of Notch in nM in the stable transfection Notch reporter assay.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

What is claimed is:

1. A compound of the structure: 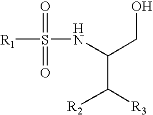
wherein:
R₁ is aryl, substituted aryl, heteroaryl, or substituted heteroaryl;
R₂ is haloalkyl or substituted haloalkyl; and
R₃ is aryl, substituted aryl, heteroaryl, or substituted heteroaryl;
or a pharmaceutically acceptable salt, prodrug, tautomer, or metabolite thereof.

2. The compound according to claim 1, wherein R₁ is a 6 to 14 membered unsaturated carbon-based ring or substituted 6 to 14 membered unsaturated carbon-based ring.

3. The compound according to claim 1, wherein R₁ is of the structure: 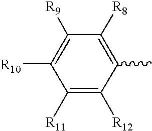
wherein:
R⁸, R⁹, R¹⁰, R¹¹, and R¹² are independently selected from the group consisting of H, halogen, C₁ to C₆ alkoxy, substituted C₁ to C₆ alkoxy, NO₂, C₁ to C₆ alkyl, substituted C₁ to C₆ alkyl, CN, C₁ to C₆ alkylcarbonyl, substituted C₁ to C₆ alkylcarbonyl, C₁ to C₆ alkylcarboxy, substituted C₁ to C₆ alkylcarboxy, CONH₂, CONH(C₁ to C₆ alkyl), CONH (substituted C₁ to C₆ alkyl), CON(C₁ to C₆ alkyl)₂, CON (substituted C₁ to C₆ alkyl)₂, S(C₁ to C₆ alkyl), S (substituted C₁ to C₆ alkyl), SO(C₁ to C₆ alkyl), SO (substituted C₁ to C₆ alkyl), SO₂(C₁ to C₆ alkyl), SO₂ (substituted C₁ to C₆ alkyl), NHSO₂(C₁ to C₆ alkyl), and NHSO₂ (substituted C₁ to C₆ alkyl); or
one or more of R⁸ and R⁹; or R⁹ and R¹⁰; or R¹¹ and R¹²; or R¹⁰ and R¹¹ are fused to form:
(i) a saturated ring containing 3 to 8 carbon atoms;
(ii) an unsaturated ring containing 5 to 8 carbon atoms; or
(iii) a heterocyclic ring containing 1 to 3 heteroatoms selected from the group consisting of O, N, and S in the backbone of the ring;
wherein rings (i) to (iii) may be substituted by 1 to 3 substituents including C₁ to C₆ alkyl, substituted C₁ to C₆ alkyl, halogen, or CN.

4. The compound according to claim 1, wherein R₁ is 4-chlorophenyl.

5. The compound according to claim 1, wherein R₁ is of the structure: 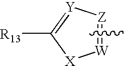
wherein:
R₁₃ is selected from the group consisting of H, halogen, and CF₃;
W, Y and Z are independently selected from the group consisting of C, CR₁₄ and N, wherein at least one of W, Y or Z is C;
X is selected from the group consisting of O, S, SO₂, and NR₁₅;
R₁₄ is selected from the group consisting of H, halogen, C₁ to C₆ alkyl, and substituted C₁ to C₆ alkyl; and
R₁₅ is selected from the group consisting of H, C₁ to C₆ alkyl, C₃ to C₈ cycloalkyl, SO₂(C₁ to C₆ alkyl), SO₂ (substituted C₁ to C₆ alkyl), SO₂aryl, SO₂ substituted aryl, CO(C₁ to C₆ alkyl), CO (substituted C₁ to C₆ alkyl), CO aryl and CO substituted aryl.

6. The compound according to claim 5, wherein R₁ is 2-chloro-thiophen-5-yl.

7. The compound according to claim 1 which has S-stereochemistry at the carbon bearing the sulfonamide nitrogen atom.

8. The compound according to claim 1, wherein:
R₂ is —(CHₘX′ₙ)_{z}CHₚX′_{q};
m and n are, independently, 0 to 2, provided that m+n=2;
p and q are, independently, 0 to 3, provided that p+q=3;
z is 0 to 12; and
X′ is halogen;
provided that both n and q are not 0.

9. The compound according to claim 1, wherein:
R₂ is —(CHₘ(R₅)_{y}X′ₙ)_{z}CHₚ(R₅)ₒX′_{q};
y, m, and n are, independently, 0 to 2, provided that y+m+n=2;
o, p, and q are, independently, 0 to 3, provided that o+p+q=3;
z is 0 to 12;
provided that both n and q are not 0.
X′ is halogen; and
R₅ is halogen, CN, OH, NO₂, C₁ to C₆ alkyl, C₁ to C₆ substituted alkyl, C₂ to C₆ alkenyl, substituted C₂ to C₆ alkenyl, C₂ to C₆ alkynyl, C₂ to C₆ substituted alkynyl, amino, aryl, substituted aryl, heterocyclic, substituted heterocyclic, heteroaryl, substituted heteroaryl, C₁ to C₆ alkoxy, aryloxy, C₁ to C₆ alkylcarbonyl, C₁ to C₆ alkylcarboxy, and arylthio.

10. The compound according to claim 9, wherein R₂ is (C₁ to C₅ alkyl)CF₃.

11. The compound according to claim 1 which has S— or R-stereochemistry at the carbon atom attached to R₂ and R₃.

12. The compound according to claim 1, wherein:
R₁ is substituted phenyl or substituted thiophene;
R₂ is CF₃;
R₃ is phenyl or phenyl substituted with one or more halogen atoms;
provided that the carbon-atom attached to the sulfonamide nitrogen atom has S-stereochemistry;
provided that the carbon atom attached to R₂ and R₃ has R-stereochemistry.

13. The compound according to claim 1 which is 5-Chloro-N-[2-(3,5-difluorophenyl)-3,3,3-trifluoro-1-(hydroxymethyl)propyl]thiophene-2-sulfonamide.

14. The compound according to claim 13, which has an X-ray diffraction peak pattern comprising a peak at 2θ of about 6.4°±0.3°.

15. The compound according to claim 14, wherein the X-ray diffraction peak pattern further comprises one or more peaks at 2θ of about 14.8°±0.3°, 16.1°±0.3°, 18.3°±0.3°, 19.1°±0.3°, 19.5°±0.3°, 22.1°±0.3°, 22.6°±0.3°, or 24.3°±0.3°.

16. The compound according to claim 1, which is 5-Chloro-N-[3,3,3-trifluoro-1-(hydroxymethyl)-2-phenylpropyl]thiophene-2-sulfonamide; 5-Chloro-N-[(1S,2R)-3,3,3-trifluoro-1-(hydroxymethyl)-2-phenylpropyl]thiophene-2-sulfonamide; 5-Chloro-N-[(1S,2S)-3,3,3-trifluoro-1-hydroxymethyl-2-phenylpropyl]thiophene-2-sulfonamide; 4-Chloro-N-[(1S,2R) -3,3,3-trifluoro-1-(hydroxymethyl)-2-phenylpropyl]benzenesulfonamide; 5-Chloro-N-[(1S,2S)-2-(3,5-difluorophenyl)-3,3,3-trifluoro-1-(hydroxymethyl)propyl]thiophene-2-sulfonamide; 5-chloro-N-[(1R,2R)-2-(3,5-difluorophenyl)-3,3,3-trifluoro-1-(hydroxymethyl)propyl]thiophene-2-sulfonamide; 5-Chloro-N-[(1S,2R)-2-(3,5-difluorophenyl)-3,3,3-trifluoro-1-(hydroxymethyl)propyl]thiophene-2-sulfonamide; 5-Chloro-N-[(1S,2R)-3,3,3-trifluoro-2-(4-fluorophenyl)-1-(hydroxymethyl)propyl]thiophene-2-sulfonamide; 5-Chloro-N-[(1S,2S)-3,3,3-trifluoro-2-(4-fluorophenyl)-1-(hydroxymethyl)propyl]thiophene-2-sulfonamide; 5-Chloro-N-[(1S,2S)-3,3,3-trifluoro-2-(3-fluorophenyl)-1-(hydroxymethyl)propyl]thiophene-2-sulfonamide; 5-Chloro-N-[(1S,2R)-3,3,3-trifluoro-2-(3-fluorophenyl)-1-(hydroxymethyl)propyl]thiophene-2-sulfonamide; 5-Chloro-N-[(1S,2R)-2-(4-chlorophenyl)-3,3,3-trifluoro-1-(hydroxymethyl)propyl]thiophene-2-sulfonamide; 5-Chloro-N-[(1S, 2S)-2-( 4-chlorophenyl)-3,3,3-trifluoro-1-(hydroxymethyl)propyl]thiophene-2-sulfonamide; or a pharmaceutically acceptable salt, prodrug, tautomer, or metabolite thereof.

17. The compound according to claim 1, wherein the pharmaceutically acceptable salt is of a base.

18. The compound according to claim 17, wherein said base is selected from the group consisting of sodium hydroxide, lithium hydroxide, potassium hydroxide, and mixtures thereof.

19. A pharmaceutical composition comprising a compound of claim 1 and a physiologically compatible carrier.

20. A pharmaceutical composition comprising a prodrug of the compound of claim 1 and a physiologically compatible carrier.

21. A method of inhibiting beta amyloid production in a subject, said method comprising delivering a compound of claim 1.

22. The method according to claim 21, wherein said compound is delivered orally, by injection, by inhalation, transdermally and suppository.

23. A method of treating a disease selected from the group consisting of Alzheimer's disease, amyloid angiopathy, cerebral amyloid angiopathy, systemic amyloidosis, hereditary cerebral hemorrhage with amyloidosis of the Dutch type, inclusion body myositis, mild cognitive impairment (MCI) and Down's syndrome, in a subject, said method comprising administering a compound of claim 1 to said subject in an amount sufficient to alleviate the symptoms or progress of said disease.

24. A pharmaceutical kit comprising a container comprising a pharmaceutical composition of claim 19.

25. A method for preparing a compound according to claim 1, wherein said method is selected from the group consisting of (i), (ii), (iii), (iv), and (v):
method (i), which comprises:
(a) reacting a halogenated acetophenone, a first base, and a tri-alkyl phosphonoacetate to form an α,β-unsaturated ester;
(b) reducing said α,β-unsaturated ester to a saturated ester;
(c) converting said saturated ester to an enolate;
(d) converting said enolate to an azido-ester;
(e) reducing said azido-ester to an amino-ester;
(f) sulfonylating said amino-ester to a sulfonamido-ester; and
(g) reducing said sulfonamido-ester;
method (ii) which comprises:
(a) hydrolyzing an α,β-unsaturated ester to an α,β-unsaturated carboxylic acid;
(b) converting said α,β-unsaturated carboxylic acid to a mixed anhydride;
(c) reacting said mixed anhydride with a nucleophile comprising a chiral auxiliary;
(d) reducing the product of step (c);
(e) reacting the product of step (d) with a base;
(f) converting the product of step (e) to an azido-imide;
(g) reducing said azido-imide to an amino-imide;
(h) sulfonylating said amino-imide to a sulfonamido-imide; and
(i) reducing said sulfonamido-imide;
method (iii), which comprises:
(a) converting a halogenated acetophenone to an α,β-unsaturated carboxylic acid;
(b) hydrogenating said an α,β-unsaturated carboxylic acid to a saturated carboxylic acid;
(c) converting said carboxylic acid to a mixed anhydride;
(d) reacting said mixed anhydride with a nucleophile comprising a chiral auxiliary;
(e) reacting the product of step (d) with a base;
(f) converting the product of step (e) to an azido-imide;
(g) reducing said azido-imide to an amino-imide;
(h) sulfonylating said amino-imide to a sulfonamido-imide; and
(i) reducing said sulfonamido-imide
method (iv), which comprises:
(a) reacting a halogenated acetophenone, an alkyl isocyanoacetate, and a base;
(b) reducing the product of step (a) with sodium borohydride in methanol;
(c) reacting the product of step (b) with lithium borohydride;
(d) hydrolyzing the product of step (c) with an acid to an amine; and
(e) sulfonylating said amine; and
method (v) which comprises:
(a) hydrolyzing an α,β-unsaturated ester to an α,β-unsaturated carboxylic acid;
(b) converting said carboxylic acid to a mixed anhydride;
(c) reacting said mixed anhydride with a nucleophile comprising a chiral auxiliary;
(d) reducing the product of step (c);
(e) reacting the product of step (d) with a base;
(f) converting the product of step (e) to an azido-imide;
(g) reducing said azido-imide to an azido-alcohol;
(h) reducing said azido-alcohol to an amino-alcohol; and
(i) sulfonylating said amino-alcohol.

26. The method according to claim 25, wherein in step (i)(a) said tri-alkyl phosphonoacetate is (R₆O)₂P(O)CH₂CO₂R₇, R₆ is C₁ to C₆ alkyl and substituted alkyl; and R₇ is C₁ to C₆ alkyl, substituted C₁ to C₆ alkyl, C₂ to C₆ alkenyl, substituted C₂ to C₆ alkenyl, C₂ to C₆ alkynyl, C₂ to C₆ substituted alkynyl, aryl, substituted aryl, heteroaryl, or substituted heteroaryl.

27. The method according to claim 25, wherein in step (iv)(a) said alkyl isocyanoacetate is CNCH₂CO₂R₁₇ and R₁₇ is C₁ to C₆ alkyl or substituted C₁ to C₆ alkyl.

28. The method according to claim 25, wherein in step (ii)(c) or step (iii)(d), said nucleophile comprising a chiral auxiliary is an oxazolidinone.

29. The method according to claim 28, wherein said oxazolidinone is:

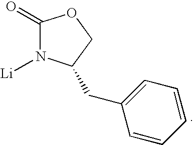

30. The method according to claim 25, wherein in step (ii)(b) said mixed anhydride is of the structure: 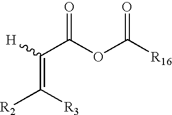
wherein:
R₁₆ is C₁ to C₆ alkyl or substituted C₁ to C₆ alkyl.

31. The method according to claim 25, wherein in step (iii)(c) said mixed anhydride is of the structure: 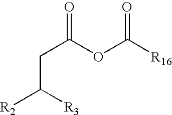
wherein:
R₁₆ is C₁ to C₆ alkyl or substituted C₁ to C₆ alkyl.

32. A method according to claim 25, wherein R₂ is trifluoromethyl.
